(19) United States
(12) Patent Application Publication
(10) Pub. No.: US 2020/0155726 A1
(43) Pub. Date: May 21, 2020

(54) A COMPOSITE, SCAFFOLD AND APPLICATIONS THEREOF

(71) Applicant: JAWAHARLAL NEHRU CENTRE FOR ADVANCED SCIENTIFIC RESEARCH, Karnataka, Bangalore (IN)

(72) Inventors: Govindaraju THIMMAIAH, Bangalore (IN); Shivaprasad MANCHINEELLA, Bangalore (IN)

(21) Appl. No.: 16/077,391

(22) PCT Filed: Feb. 10, 2017
(86) PCT No.: PCT/IB2017/050734

§ 371 Date: Aug. 10, 2018

(30) Foreign Application Priority Data
Feb. 10, 2016 (IN) 201641004770

(51) Int. Cl. A61L 27/48; A61L 27/56; C07K 14/78; C07K 1/14

(52) U.S. Cl. CPC A61L 27/48; C07K 1/14; C07K 14/78; A61L 27/56

(57) ABSTRACT

The present disclosure relates to the field of biomaterials, tissue engineering and regenerative medicine. Particularly, the present disclosure relates to biomaterial composite and preparation of various scaffold formats using the composite. The disclosure provides for preparation of biomaterial composite comprising silk fibroin and melanin, preparation of scaffolds with the biomaterial composite and its applications in tissue engineering, electrotherapy applications and regenerative medicine. In some embodiments, the silk fibroin is a peptide modified silk fibroin. The biomaterial composite of the present disclosure exhibits various advantages including but not limiting to enhanced antioxidant property, superior electroactive properties, improved myogenic cell differentiation, better cell growth and regeneration, and enhanced cell adhesion property.

### TECHNICAL FIELD

The present disclosure relates to the field of biomaterials, tissue engineering and regenerative medicine. Particularly, the present disclosure relates to biomaterial composite and preparation of various scaffold formats using the composite. The disclosure provides for preparation of biomaterial composite comprising silk fibroin and melanin, preparation of scaffolds with the biomaterial composite and its applications in tissue engineering, electrotherapy applications and regenerative medicine. In some embodiments, the silk fibroin is a modified silk fibroin with peptide.

### BACKGROUND AND PRIOR ART OF THE DISCLOSURE

Any discussion of the prior art throughout the specification should in no way be considered as an admission that such prior art is widely known or forms part of common general knowledge in the field.

The synthetic polymers used for tissue engineering and regenerative medicine applications have limitations associated with their biocompatibility, immunogenic response from tissues due to the development of oxidative stress at biomaterial interface. Apart from the biocompatibility, the properties of scaffolds made from synthetic polymers vary from batch to batch and needs extra bio-functionalization to avoid immunogenic response from the host immune system.

Biomaterial scaffolds with desired physical properties such as stiffness, topography, magnetic and conducting properties along with cell adhesion and proliferation cues finds potential use for tissue engineering and regenerative medicine applications. Scaffolds that are biocompatible, biodegradable and bioresorbable along with other desirable physical and chemical properties are most sought after for both in vitro and in vivo applications. The soft biomaterial scaffolds, which are made from synthetic polymers, suffer the limitations such as the toxicity associated with their synthesis or degradation, differences in batch-to-batch integrity of polymers and the requirement of additional biofunctionalisation to facilitate cell adhesion and proliferation. Biomaterial scaffolds that are made from materials of biological origin address the limitation associated with synthetic polymer based scaffolds. However, the industrial scale availability, stability and ease of processability and moldability into different scaffold formats limit the use of many biopolymers largely.

Electrically active and conducting biomaterial scaffolds have drawn enormous attention in tissue engineering and regenerative medicine applications for tissues, whose functions are highly coordinated by endogenously generated electric fields (bioelectricity). Cardiac myocytes, skeletal myoblasts, neurons and osteoblasts are the examples of cells that respond to electric stimuli. Another crucial aspect that needs to be addressed at the host tissue-biomaterial interface is the development of oxidative stress by cells and tissues, which leads to cell death and tissue injury and maximizes inflammation. Moreover, oxidative stress is also associated with several pathological conditions and delayed wound healing.

Skeletal muscle tissue engineering involves the design of biomaterial scaffolds that can promote myogenic differentiation in myoblasts to generate functional myotubes and offers potential applications in repairing and regenerating the impaired muscle tissues. Developing such scaffolds has potential applications both in understanding the myotube assembly process in vitro and in regenerating the damaged muscle tissue in vivo. Skeletal myoblasts are electroactive in nature and the myogenic differentiation into myotubes can be modulated using electroactive biomaterial scaffolds, when coupled with optimal signaling molecules (biochemical cues). Mimicking the extracellular matrix of muscle cells in designing novel biomaterial scaffolds for skeletal muscle tissue engineering has drawn the attention of several researchers.

Synthetic conducting polymers (CPs) such as polyaniline (PANi), polypyrrole (PPy), poly(3,4-ethylenedioxythiophene) (PEDOT), and multi-wall carbon nanotubes (MWNTs) mixed with synthetic and natural polymers in electrospun scaffold form and bioceramics with conducting properties have been used for promoting myogenic differentiation in vitro. In spite of the promising applications in modulating cellular functions, CPs have limited use for long-term in vivo tissue engineering (TE) applications because of the problems of biocompatibility, toxicity and non-biodegradability along with difficulty in fabricating different 3D scaffold formats. Further, the scaffolds prepared from the CP-synthetic polymer blends require additional biofunctionalization with biomolecules to make the scaffolds suitable for cell adhesion and proliferation.

The present disclosure overcomes the limitations of the products and methods of the prior art by providing an improved biomaterial composite and corresponding methods and applications. The present disclosure overcomes the aforesaid limitations such as biocompatibility, toxicity, non-biodegradability, immunogenic response and difficulty in fabricating different 3D scaffold formats.

### BRIEF DESCRIPTION OF THE ACCOMPANYING FIGURES

In order that the disclosure may be readily understood and put into practical effect, reference will now be made to exemplary embodiments as illustrated with reference to the accompanying figures. The figures together with a detailed description below, are incorporated in and form part of the specification, and serve to further illustrate the embodiments and explain various principles and advantages, in accordance with the present disclosure wherein:

FIG. 1 illustrates the schematic of silk fibroin extraction from B. mori cocoons.

FIG. 2 illustrates the preparation and modification of SFFs.

FIG. 3 illustrates the purification and characterization of YIGSR peptide.

FIG. 3A illustrates HPLC chromatogram and

FIG. 3B illustrates HRMS spectra (HPLC: High performance liquid chromatography; HRMS: high resolution mass spectrometry).

FIG. 4 illustrates the purification and characterization of GYIGSR peptide.

FIG. 4A illustrates HPLC chromatogram and

FIG. 4B illustrates HRMS spectra.

FIG. 5 illustrates the chemical structures of laminin derived peptides YIGSR and GYIGSR.

FIG. 6 illustrates the surface morphology analysis. SEM micrographs of pristine and modified SFFs. SEM: scanning electron microscopy.

FIG. 7 illustrates the contact angle values and images of water and DMEM droplets on the surface of SF and SM spin coated scaffolds.

FIG. 8 illustrates the fabrication and characterization of scaffolds.

8A) illustrates the Scanning electron microscopy (SEM) micrographs of A1) SF mat with diameter 470±45 nm and A2) SM mat with diameter 343±40 nm. Insets in A1) and A2) show the 6 mm discs cut from the corresponding electrospun mats for cell culture experiments.

8B) illustrates the FT-IR analysis of as-prepared and methanol-treated films and mats. Broken lines represent as-prepared scaffolds while solid lines represent methanol-treated scaffolds. Vertical dotted lines are inserted to visualize shift in the absorption before and after methanol treatment.

8C) and 8D) illustrate the thermogravimetric (TG) and the differential scanning calorimetry (DSC) analysis of SF, SM films and mats.

8E) illustrates the sheet resistance values of scaffolds measured under physiological conditions.

8F) illustrates the free radical scavenging property of SF, SM solutions and scaffolds (films and mats) against DPPH free radicals.

8G) illustrates the Cellular reactive oxygen species detection assay. Fluorescence from myoblasts cultured on TCPS control, SF film, SM film, SF mat and SM mat after 48 hours of culture and treatment with CM-H2DCFDA. Fluorescence monitored at 535 nm with the excitation at 485 nm.

FIG. 9 illustrates the Scanning electron microscopy (SEM) micrographs of:

9A) SF film and

9B) SM films.

9C) and 9D) illustrates the photographs of SS mesh modified stationary collector and free standing electrospun mats (SM and SF) respectively.

FIG. 10 illustrates the cell viability and proliferation studies of myoblasts on scaffolds.

10A) illustrates the MTT assay results showing number of viable cells on SF film, SM film, SF mat and SM mat samples cultured for a time period of 1, 2 and 3 days, respectively. ‘*’ denotes statistically significant difference (p<0.05) in cell number with respect to the corresponding control within each group. ‘#’ denotes significant difference compared with day 1 of each samples and indicates significant difference between films and electrospun mat at the given time point. Data points represent mean±SD (n=3).

10B) illustrates the Live/dead staining of C2C12 myoblasts grown on B1) TCPS, B2) SF film, B3) SF mat B4) SM film and B5) SM mat after 3 days of culture. (Green=FDA (live); red=PI (dead)); scale bar=100 μm.

10C) illustrates the PicoGreen assay results showing total DNA content of cells on TCPS, SF film, SM film, SF mat and SM mat samples cultured for a time period of 1, 2 and 3 days, respectively. ‘#’ denotes significant difference compared with day 1 of each sample. Data points represent mean±SD (n=3). Standard curve of known dsDNA (ng/mL) is used to calculate the DNA content from the samples.

FIG. 11 illustrates the myogenic differentiation of C2C12 myoblasts and quantification of myotube formation on scaffolds.

11A) illustrates the SEM micrographs of myoblasts on A1) TCPS, A2) SF film, A3) SM film, A4) SF mat, A5) SM mat after 7 days of culture (3 days in serum starvation) and A6) shows the magnified image of myotubes on SM mat.

11B) illustrates the average length and width of myotubes on SF and SM scaffolds after 3 days of differentiation are calculated using corresponding SEM micrographs.

FIG. 12 illustrates the fluorescence staining of myoblasts on control, SF film, SF mat, SM film and SM mat after 3 days in differentiation medium (serum starvation). Actin filaments and nucleus are stained with Alexa Fluor 488-Phalloidin (green) and Hoechst stain (blue) respectively. Scale bar 100 μm.

FIG. 13 illustrates FT-IR and TG.

FIG. 13A illustrates FT-IR; dashed vertical lines in 6A indicates the amide I and II positions characteristic to silk β-sheet conformation.

FIG. 13B illustrates TG analysis of pristine and modified SFFs. (FTIR: Fourier transform infrared spectroscopy; TG: thermo gravimetry).

FIG. 14 illustrates the surface roughness analysis. AFM micrographs of pristine and YIGSR covalently functionalized (SFF CL1) SFFs. (AFM: atomic force microscopy)

FIG. 15 illustrates the cytocompatibility of SFFs. MTT assay results showing number of viable cells on pristine and modified SFFs after 1, 3, 7 days of culture respectively.

FIG. 16 illustrates the fluorescence staining of hMSCs on control, pristine and modified SFFs after culturing in differentiation medium (added retinoic acid). Actin filaments and nucleus are stained with Alexa Fluor 488-Phalloidin, Hoechst stain giving green and blue fluoresce respectively.

### STATEMENT OF THE DISCLOSURE

Accordingly the present disclosure relates to a biomaterial composite comprising silk protein and melanin; a method of preparing a biomaterial composite comprising silk protein and melanin comprising steps of—a) obtaining silk protein sponges; and b) treating the silk protein sponges with melanin in presence of a solvent to obtain the composite; a biomaterial scaffold comprising the composite as described above; a peptide modified silk fibroin, wherein the peptide is a laminin derived peptide selected from a group comprising YIGSR, GYIGSR and a combination thereof, and wherein the peptide optionally comprises an amino acid linker; a method of preparing peptide modified silk fibroin, said method comprising physically adsorbing the peptide on the silk fibroin or covalent modification of the silk fibroin with the peptide wherein said covalent modification comprises covalent peptide bond formation with N-terminus of the peptide to aspartic acid, glutamic acid side chain acid groups of the silk fibroin, or a combination thereof; a method of repairing or regenerating a biological tissue, said method comprising contacting the biomaterial composite, or the biomaterial scaffold, or the peptide modified silk fibroin as described above with the existing tissue or cells; and a method of treating a medical condition, said method comprising implanting the biomaterial scaffold, or a biomaterial scaffold comprising the peptide modified silk fibroin as described above into the subject.

### DETAILED DESCRIPTION OF THE DISCLOSURE

The present disclosure relates to a biomaterial composite comprising silk protein and melanin.

In an embodiment of the present disclosure, the silk protein is silk fibroin, spider silk or a combination thereof, preferably silk fibroin.

In another embodiment of the present disclosure, the silk fibroin and the melanin are in amount ranging from about 95:05 (wt/wt) to 80:20 (wt/wt), preferably 90:10 (wt/wt).

In yet another embodiment of the present disclosure, the silk fibroin is optionally modified with peptide.

In still another embodiment of the present disclosure, the peptide is a laminin derived peptide selected from a group comprising YIGSR, GYIGSR and a combination thereof; and wherein the peptide optionally comprises a linker, preferably amino acid.

In still another embodiment of the present disclosure, the composite is in a format selected from a group comprising film, mat, membrane, gel, sponge and combinations thereof.

The present disclosure also relates to a method of preparing a biomaterial composite comprising silk protein and melanin comprising steps of:
a) obtaining silk protein sponges; and
b) treating the silk protein sponges with melanin in presence of a solvent to obtain the composite.

In an embodiment of the present disclosure, the silk protein is silk fibroin; and wherein the silk fibroin and melanin are in amounts ranging from about 95:05 to 80:20.

In another embodiment of the present disclosure, the method is carried out at a temperature ranging from about 25° C. to 30° C., and for a time period ranging from about 48 hours to 96 hours.

In yet another embodiment of the present disclosure, the solvent is selected from a group comprising Hexafluoro-2-propanol (HFIP), formic acid and a combination thereof.

In still another embodiment of the present disclosure, the method as described above, wherein the composite is in a format selected from a group comprising film, mat, membrane, gel, sponge and combinations thereof; wherein the composite film is obtained by drop casting the composite solution obtained in step (b) of the above method followed by treatment with a solvent and drying; and wherein the composite mat is obtained by electrospinning the composite solution obtained in step (b) of the above method.

In still another embodiment of the present disclosure, the silk protein is optionally modified with peptide; and wherein the peptide modified silk protein is prepared by physical adsorption of the peptide or covalent modification of the silk protein with the peptide wherein said covalent modification comprises covalent peptide bond formation with N-terminus of the peptide to aspartic acid, glutamic acid side chain acid groups of the silk protein, or a combination thereof.

The present disclosure also relates to a biomaterial scaffold comprising the composite as described above.

The present disclosure further relates to a peptide modified silk fibroin, wherein the peptide is a laminin derived peptide selected from a group comprising YIGSR, GYIGSR and a combination thereof; and wherein the peptide optionally comprises an amino acid linker.

The present disclosure further relates to a method of preparing peptide modified silk fibroin, said method comprising physically adsorbing the peptide on the silk fibroin or covalent modification of the silk fibroin with the peptide wherein said covalent modification comprises covalent peptide bond formation with N-terminus of the peptide to aspartic acid, glutamic acid side chain acid groups of the silk fibroin, or a combination thereof.

In an embodiment of the present disclosure, the biomaterial composite, or the biomaterial scaffold, or the peptide modified silk fibroin as described above for use as a medicament.

In another embodiment of the present disclosure, the biomaterial composite or the biomaterial scaffold or the peptide modified silk fibroin as described above, for use in cell differentiation, tissue engineering, regenerative medicine and electrotherapy.

The present disclosure further relates to a method of repairing or regenerating a biological tissue, said method comprising contacting the biomaterial composite, or the biomaterial scaffold, or the peptide modified silk fibroin as described above with the existing tissue or cells.

In an embodiment of the present disclosure, the tissue or cells are selected from a group comprising stem cells, brain cells, cranial tissue, nerve tissue, spinal disc, lung, cardiac muscle, skeletal muscle, bone, cartilage, tendon, ligament, liver, kidney, spleen, pancreas, bladder, pelvic floor, uterus, blood vessel, breast, skin and combinations thereof.

The present disclosure further relates to a method of treating a medical condition, said method comprising implanting the biomaterial scaffold, or a biomaterial scaffold comprising the peptide modified silk fibroin as described above into the subject.

In an embodiment of the present disclosure, the medical condition is selected from a group comprising rheumatic disease, neurodegenerative disease, cardiovascular disease, wound healing and combinations thereof.

The present disclosure overcomes the non-limited drawbacks of the prior art, a biomaterial composite and scaffold possessing biocompatibility, antioxidant, electroactive and free radical scavenging activities is provided, wherein said biomaterial composite comprises silk protein and melanin.

In an embodiment, the silk protein is silk fibroin.

In an embodiment of the present disclosure, the silk fibroin is modified/surface functionalized with laminin β1 derived integrin binding short peptides with or without a flexible glycine linker at the N-terminus. In an exemplary embodiment, the silk fibroin is modified with the peptide—YIGSR, its modified analogue—GYISR, or a combination thereof.

In other embodiments, other silk proteins may also be employed in the biomaterial composite including but not limiting to spider silk.

The present disclosure further provides biomaterial scaffolds comprising the above mentioned composite comprising silk protein and melanin.

The present disclosure primarily relates to a biomaterial composite comprising silk fibroin protein and melanin.

The present disclosure further relates to a biomaterial composite comprising silk fibroin and melanin, wherein the silk fibroin is modified with peptide. In an exemplary embodiment, the silk fibroin is modified with the peptide—YIGSR, its modified analogue—GYISR, or a combination thereof.

The present disclosure relates to a biomaterial composite comprising silk fibroin protein and melanin in any ratio, percentage or quantity.

In an embodiment, the biomaterial composite comprises silk fibroin and melanin at a concentration ranging from about 95:05 to 80:20.

In a preferred embodiment, the biomaterial composite comprises silk fibroin and melanin at a concentration of about 90:10 (silk fibroin: melanin) wt/wt.

In an embodiment of the present disclosure, the silk fibroin is prepared by steps comprising:
cutting cocoons into about 4 cm×4 cm size pieces;
boiling the cocoon pieces for about 30 min in Na₂CO₃ solution to remove water-soluble sericin;
washing the resulting native silk fibroin fibers thoroughly with plenty of water to remove the water soluble sericin outer layer and drying;

II
dissolving the obtained native silk fibroin fibers in LiBr at about 60° C. for about 4 hours;
dialyzing the obtained amber-colored solution using an activated cellulose membrane against water;
subjecting the aqueous regenerated silk fibroin solution to centrifugation for about 20 min at about 6000 rpm, at about 4° C. to remove the insoluble debris and stored at about 4° C. until further use; and
lyophilizing aqueous fibroin solution by initially freezing the solution using liquid N₂, followed by freeze drying at about −70° C. for about 14 hours to yield water-insoluble fibroin sponges.

The present disclosure also provides a method of preparing a biomaterial composite film comprising silk fibroin and melanin comprising steps of:
preparing silk fibroin sponges;
dissolving the silk fibroin sponges in a solvent along with melanin at a concentration of silk fibroin sponges: melanin ranging from about 95:05 to 80:20 to obtain a solution;
drop casting the above solution on a substrate followed by treatment with alcohol and drying under high vacuum to obtain the silk-melanin composite films.

In an embodiment, the vacuum dried films are stored in air tight bags at room temperature until further use.

In an embodiment, the silk fibroin sponges of the above method is reconstituted with a solvent along with melanin, wherein the solvent is selected from a group comprising Hexafluoro-2-propanol (HFIP) and formic acid or a combination thereof. In a preferred embodiment, the silk fibroin sponges are dissolved in HFIP along with melanin at a concentration of silk fibroin sponges: melanin ranging from about 95:05 to 80:20 to obtain a solution.

In an embodiment, the substrate is polystyrene plate.

In an embodiment, the alcohol is methanol.

In an exemplary embodiment, the silk fibroin films are treated with methanol-water mixture at a concentration of about 90% and air dried overnight at a temperature ranging from about 25° C. to 30° C. for a time period ranging from about 48 hours to 96 hours to obtain methanol treated silk fibroin films.

In an embodiment of the present disclosure, the silk fibroin of the above method is modified with peptides.

In an exemplary embodiment of the present disclosure, the modified silk fibroin comprises silk fibroin and peptides in any ratio, percentage or quantity.

In another exemplary embodiment of the present disclosure, the silk fibroin is covalently modified with peptides, or peptides are physically adsorbed on silk fibroin, or both covalent modification and physical adsorption is performed.

In an embodiment, the modified silk fibroin is prepared through physical adsorption of peptides and covalent peptide bond formation between N-terminus of peptides and aspartic acid, glutamic acid side chain acid groups of silk fibroin.

In an exemplary embodiment of the present disclosure, modified silk fibroin is prepared through physical adsorption of peptides, covalent attachment of peptides or a combination thereof.

In an embodiment of the present disclosure, silk fibroin is modified with laminin derived peptide.

In an exemplary embodiment of the present disclosure, silk fibroin is modified with 131 laminin derived peptide.

In another exemplary embodiment, silk fibroin is modified with laminin (31 derived integrin binding short peptides with or without a flexible glycine linker at the N-terminus.

In yet another exemplary embodiment of the present disclosure, the laminin derived peptide is YIGSR.

In yet another exemplary embodiment of the present disclosure, the β1 laminin derived peptide and its modified analogue are employed for modifying biomaterial film comprising silk fibroin.

In yet another exemplary embodiment of the present disclosure, the laminin derived peptide is YIGSR and its modified analogue is GYIGSR.

In an embodiment, the silk fibroin is modified with peptides selected from a group comprising YIGSR, GYIGSR, and a combination thereof.

In an embodiment, the present disclosure also provides for synthesis and characterization of peptides which are employed to prepare modified silk fibroin.

In an embodiment, Fmoc-Rink amide resin is used as solid support and O-(benzotriazol-1-yl)-N, N, N′, N′-tetramethyluronium hexafluorophosphate along with N, N-diisopropylethylamine as activation mixture for the synthesis of peptides.

In still another exemplary embodiment, the silk fibroin is treated with solvent before modification with peptides, wherein the solvent is selected from a group comprising HFIP, formic acid and a combination thereof. In a preferred embodiment, the silk fibroin is dissolved in about 4 wt % HFIP at a temperature ranging from about 25° C. to 30° C.

In an exemplary embodiment, the silk fibroin films are treated with methanol-water mixture at a concentration of about 90% and air dried overnight at a temperature ranging from about 25° C. to 30° C. to obtain methanol treated silk fibroin films with β-sheet formation.

In an embodiment, the silk fibroin films with β-sheet formation are prepared and modified with laminin (31 derived integrin binding short peptides in presence of an optional linker or spacer. Said linker or spacer is used for better cell interaction of bioactive peptide motifs.

In an embodiment, the optional linker or spacer includes but is not limiting to an amino acid used for better cell interaction of bioactive peptide motifs. In a non-limiting embodiment, the amino acid used as a linker or spacer for better cell interaction of bioactive peptide motifs is glycine.

In an exemplary embodiment, silk fibroin films are prepared and modified with laminin β1 derived integrin binding short peptides along with an optional linker or spacer for better cell interaction of bioactive peptide motifs not limiting to N-terminus.

In another exemplary embodiment, silk fibroin films are prepared and modified with laminin β1 derived integrin binding short peptides along with an optional linker or spacer for better cell interaction of bioactive peptide motifs, wherein such modification or attachment of peptides onto silk fibroin via. optional linker/spacer is carried out through physical adsorption of peptides and covalent peptide bond formation between N-terminus of peptides and aspartic acid side chain acid group of silk fibroin.

In another exemplary embodiment, silk fibroin films are prepared and modified with laminin β1 derived integrin binding short peptides along with an optional linker or spacer for better cell interaction of bioactive peptide motifs, wherein such modification or attachment of peptides onto silk fibroin via. optional linker/spacer is carried out through physical adsorption of peptides and covalent peptide bond formation between N-terminus of peptides and aspartic acid, glutamic acid side chain acid group of silk fibroin.

In an exemplary embodiment, silk fibroin films (SFFs) are prepared by steps comprising:
dissolving silk fibroin sponges in a solvent such as HFIP at room temperature or formic acid at about 60° C. to obtain SFFs;
drop-casting the SFFs under ambient temperature and humidity;
air-drying the drop casted films over night at about 25° C.; and
treating the dried films with methanol-water mixture and air drying over night at room temperature to promote the β-sheet formation and to increase the stability of films in water and in culture media.

In an embodiment, physical modification, chemical modification or a combination thereof is carried out using water stable SFFs obtained after the above described methanol treatment.

In another embodiment, the peptides 1 (YIGSR) and 2 (GYIGSR) employed for modification of silk fibroin are prepared using solid phase peptide synthesis (SPPS) and are purified and characterized using high performance liquid chromatography (HPLC) and high resolution mass spectrometry (HRMS) respectively.

In an exemplary embodiment, SFFs are covalently modified by steps comprising:
soaking SFFs in phosphate buffer saline (PBS) for about 30 min to achieve the surface realignment of hydrophilic groups and to promote the covalent functionalization;
activating —COOH group of Asp and Glu amino acids by treating PBS soaked SFFs with activation buffer containing 1-ethyl-3-(dimethylaminopropyl) carbodiimide hydrochloride (EDC.HCl)/N-hydroxysuccinimide (NHS) solution for about 15 min at ambient conditions to obtain the amine reactive NHS esters of Asp and Glu side chain —COOH groups;
reacting the activated NHS ester SFFs with peptides 1 (YIGSR) and 2 (GYIGSR) in PBS (pH 7.4) at ambient temperature for about 2 hours; and
washing SFFs with PBS (pH 7.4) for about 5 min, rinsing to remove the buffer slats from the film surface and drying in vacuum at room temperature.

In an embodiment, peptides are physically adsorbed on SFFs by steps comprising:
contacting the films with laminin derived integrin binding peptides 1 (YIGSR) and 2 (GYIGSR) and whole laminin for about 4 h at about 37° C.; and
rinsing the films twice each with PBS and milli-Q water, and dried in vacuum at room temperature.

In an embodiment, crack-free, uniform silk fibroin films and modified with custom synthesized laminin β1 derived short peptides is prepared. The addition of peptide epitopes on the surface of SFFs provides a platform with cues for cell adhesion and proliferation. SFFs covalently functionalized with GYIGSR promotes cell adhesion, proliferation of hMSCs and facilitates the hMSCs differentiation into neuronal lineage, highlighting the significance of both additional flexible linker and permanent covalent modification for stem cell differentiation. The present disclosure highlights the effect of flexible linker and nature of modification of SFFs with laminin β1 sequence for modulating the differentiation fate of hMSCs.

In an exemplary embodiment, the biomaterial composite comprising silk fibroin and melanin is formulated into biomaterial scaffolds including but not limited to films, membranes, electrospun mats or combinations thereof.

In an embodiment, electrospun mats from melanin-blended silk fibroin are prepared using robust electrospinning technique. Electrospinning has versatility, compatibility with a wide range of synthetic and biopolymers and solvent systems, as well as easy scalability. The key electrospinning working parameters such as solvent, concentration, and composition (collectively known as solution parameters), voltage, needle diameter, flow rate, collector type and collector distance from the tip of the needle (collectively known as process parameters), and temperature and humidity (collectively known as ambient parameters) are optimized for the preparation of bead-free, uniform and reproducible fiber mats.

In an embodiment, techniques selected from a group comprising scanning electron microscopy (SEM), TEM (transmission electron microscope), AFM (Atomic Force Microscope) and combinations thereof is performed for analyzing the morphology of silk fibroin and melanin composite, or, silk fibroin and melanin composite wherein the silk fibroin is modified with peptide.

In an embodiment, SEM analysis of silk fibroin (SF) electrospun mats reveal the presence of bead-free, randomly aligned porous submicron diameter fiber networks. The fibers are obtained with very narrow size distribution and has an average diameter of 470±45 nm. Similarly, silk-melanin (SM) electrospun fiber mats are obtained with uniformly distributed network of fibers with narrow size distributions. The formation of relatively aligned fiber mats is observed with silk-melanin blend solution under similar conditions. The formation of bead-free and predominantly aligned fiber mats with relatively smaller fiber diameter (343±40 nm) from SM is attributed to the increased conductivity of the electrospinning solution with melanin blending. The electrospun mats are flexible for cutting or punching to attain the required scaffold dimensions for tissue engineering applications.

In another embodiment, FTIR spectrum of as-prepared SF films show characteristic peaks at 1650 cm⁻¹, 1621 cm⁻¹ in amide-I region (C═O stretching), and 1514 cm⁻¹ in amide-II region (N—H stretching), indicating the coexistence of random helical and β-sheet conformations in the films. SF films, after methanol treatment, show shifted peaks at 1699 cm⁻¹, 1619 cm⁻¹ and 1512 cm⁻¹ assigned to β-sheet conformation of silk (silk-II).

In an embodiment, vibrational frequency shift of characteristic amide-I and II peaks from random coil and helical conformation to β-sheet conformation is observed for SM films, and SF and SM mats with methanol treatment.

In an embodiment, FTIR analysis suggests trifling interference of melanin incorporation in the secondary structure of silk fibroin in scaffolds, thereby maintaining the structural stability of silk fibroin in the scaffolds comprising silk fibroin and melanin composite.

In an embodiment, the hydrophilicity of the silk fibroin and silk-melanin composite materials is assessed by contact angle measurement using milli-Q water and DMEM cell culture media under ambient conditions, using the sessile drop method.

In an embodiment, the surface morphology and roughness of the drop-casted films with silk fibroin modified with peptides is measured using Atomic force microscopy (AFM).

In an embodiment, the thermal decomposition behavior of the SF and SM films and electrospun fiber mats/scaffolds (SF mat and SM mat) is evaluated using thermo gravimetric (TG; Mettler, TGA/DSC 2) and differential scanning calorimetric (DSC; TA DSC, Q2000) analysis. In another embodiment, the thermal decomposition behavior of the pristine and modified silk films is studied by thermo gravimetric (TG) analysis. In still another embodiment, the effect of functionalization on thermal stability of silk fibroin is evaluated using thermo gravimetric (TG; Mettler, TGA/DSC 2) analysis.

In an embodiment, the decomposition temperature of melanin-incorporated films and electrospun mats is higher than the corresponding pristine SF scaffolds evidencing that the melanin-incorporated silk fibroin composites of the present disclosure are highly stable. The higher thermal stability is attributed to the increased stability of scaffolds with the incorporation of melanin with the aromatic backbone.

In another embodiment, the higher thermal stability of melanin-incorporated scaffolds is further confirmed by carrying out DSC analysis. DSC curves show higher degradation temperature for SM film and SM mat at about 280° C. and about 284° C., respectively, than that of the SF film and SF mat at 278° C. and 282° C., respectively, thus evidencing high thermal stability of the present SM composite.

In an embodiment, the electrical conducting property of silk and melanin composites in comparison with silk alone is evaluated using a two-point resistivity probe at about 25° C. and under humid conditions.

In another embodiment, the improved conductivity (i.e. decreased sheet resistance) values of the scaffolds containing melanin establishes the higher efficacy of conducting biomaterial scaffolds by blending melanin with silk fibroin.

In an embodiment, the radical scavenging capacity of silk-melanin composite and scaffolds is evaluated. In another embodiment, the radical scavenging capacity of silk-melanin composite and scaffolds is evaluated against the inhibition of lipophilic radicals such as 1,1-diphenyl-2-picrylhydrazyl (DPPH).

In an exemplary embodiment, the highest radical scavenging activity is shown by silk-melanin solutions with about 88% inhibition.

In another exemplary embodiment, the radical scavenging activity of the biomaterial scaffolds, preferably SM mats and SM films is about 68% and about 46% of radical inhibition.

Thus, silk-melanin solution shows better antioxidant activity owing to the ready accessibility of melanin to scavenge the radicals. However, since the solution as such cannot be used for the intended applications including biomaterial platform for tissue engineering, silk-melanin (SM) mats and SM films showed excellent antioxidant activity compared to the pristine SF films and mats in a sustained manner. The intriguing sustained antioxidant activity is favorable for applications including tissue engineering compared to immediate or burst activity as seen with SM solutions.

In an embodiment, the biocompatibility of scaffolds for muscle tissue engineering is evaluated by measuring the viability, cytotoxicity and proliferation of myoblasts seeded onto the scaffolds through assays selected from a group comprising MTT, LIVE/DEAD and cell proliferation assays.

In an embodiment, the viability of cells grown on the scaffolds is evaluated for their mitochondrial reductase activity of live cells using MTT assay for silk fibroin and melanin composite.

In an embodiment, the suitableness of scaffolds of silk fibroin modified with peptide and melanin composite for tissue engineering and regenerative medicine applications is evaluated by measuring the viability of hMSCs seeded on to the films through MTT assay.

In an embodiment, all the SFFs scaffolds show good cytocomaptibility and no significant cell death is observed on any silk scaffold with respect to TCPs control after day 1. A consistent and statistically significant increase in the number of viable hMSCs as compared to the TCPs control is observed for both 72 and 168 h cultures. The markedly increased cell number on SFFs on day 7 indicates that the scaffolds promote hMSCs adhesion and proliferation to a great extent.

In an embodiment, the cytotoxicity of pristine and silk-melanin composite scaffolds (films and mats) on myoblasts is calculated by LIVE/DEAD assay.

In another embodiment, Fluorescein diacetate (FDA) and propidium iodide (PI) dye combination is used for staining the live and dead cells, respectively.

In a preferred embodiment, a high fraction (>99%) of FDA-stained cells, with only few nuclei stained with PI, is observed on the scaffolds, indicating excellent biocompatibility of the scaffolds.

In an embodiment, the proliferation of myoblasts is evaluated by quantifying the total DNA content of cells cultured on pristine and composite silk scaffolds after 24, 48 and 72 hours using picogreen assay.

In another embodiment, a marked increase in the DNA content of myoblasts grown on each scaffold after 24, 48 and 72 hours is observed. There is no significant difference in the DNA content of cells grown on all the scaffolds, indicating the excellent cyto-compatibility of scaffolds.

In an embodiment, the protective role of SM scaffolds against the oxidative stress and consequent lethal effect on myoblast culture and differentiation is evaluated by measuring the intracellular reactive oxygen species (ROS) levels of myoblasts cultured on silk scaffolds using 5-(and-6)-chloromethyl-2′,7′-dichlorodihydrofluorescein diacetate (CM-H2DCFDA) assay.

In a preferred embodiment, the reduction of cellular oxidative stress in the myoblasts cultured on SF and SM scaffolds is estimated using 5-(and-6)-chloromethyl-2′,7′-dichlorodihydrofluorescein diacetate (CM-H2DCFDA) ROS indicator dye (Invitrogen).

In another embodiment, the decrease in DCFDA fluorescence on SM scaffolds (films and mats) asserts the protective antioxidant activity of melanin/silk fibroin scaffolds against the basal ROS.

In another exemplary embodiment, the protective role of biomaterial composite scaffolds against the oxidative stress and consequent lethal effect on myoblast culture and differentiation is evaluated by measuring the intracellular reactive oxygen species (ROS) levels of myoblasts cultured on silk scaffolds using 5-(and-6)-chloromethyl-2′,7′-dichlorodihydrofluorescein diacetate (CM-H2DCFDA) assay.

In an embodiment, differentiation of hMSCs on modified SFFs is studied in the presence of neuronal lineage inducing biochemical cue, retinoic acid (RA). The formation of neural like cells on the pristine and modified SFFs is analyzed by observing the morphological features of hMSCs on different SFFs using fluorescence microscope after staining the actin cytoskeleton. The results indicate that, the addition of a flexible linker in the form of Gly to the N-terminus of lamnin β1 sequence has a profound effect in driving the hMSCs differentiation into neuronal lineage similar to the whole laminin protein. Further, covalently attached GYIGSR(L2) show the significance of covalent modification in controlling the differentiation fate of stem cells. This highlights the superiority and long term utility of covalently modified silk films. In another exemplary embodiment, the present disclosure provides biomaterial composite for application in tissue engineering.

In a non-limiting embodiment, the present disclosure provides biomaterial composite comprising silk fibroin and melanin, and scaffolds made of said biomaterial composite for application in tissue engineering.

In an exemplary embodiment, the present disclosure relates to biomaterial composite of silk fibroin and melanin and its use in preparation of scaffolds for re-generation of tissue not limited to electrically excitable cardiac myocytes, skeletal neurons and osteoblasts.

The scaffold material for skeletal muscle tissue engineering should be biocompatible, biodegradable, moldable to various desired material formats and should be readily available for large-scale applications. Silk fibroin, extracted from silkworm cocoons is characterized by its superior biocompatibility, biodegradability and easy processability into various scaffold formats and is hence the base polymer for making scaffolds along with melanin in the present disclosure.

The conducting properties is imparted to the otherwise non-conducting silk scaffolds with melanin incorporation. Melanin is used specifically because of its improved biocompatibility, biodegradability and conducting properties along with its excellent antioxidant property under physiological conditions. Melanin is the naturally occurring polymeric pigment having functionalities ranging from structural coloration to protection from sunlight and radicals. Chemically, melanins are heterocyclic in nature and contain repeating units of 5,6-dihydroxyindole and 5,6-dihydroxyindole-2-carboxylic acid formed through the oxidation of tyrosine.

In the present disclosure, the strategy of combining silk fibroin and melanin wherein the silk fibroin can be optionally modified with peptides to develop antioxidant and electroactive biomaterial scaffolds for skeletal muscle tissue engineering applications in order to overcome the limitations associated with scaffolds made from blends of synthetic and conducting polymers is illustrated. Herein, biomaterial scaffold films and electrospun fiber mats are constructed using silk and melanin composite (SM) material wherein the silk fibroin can be optionally modified with peptides as described in the present disclosure with characteristic intrinsic antioxidant and conductivity properties in order to study the effect of topography along with conducting nature of the scaffold on myogenesis. The results of murine skeletal myoblast C2C12 cell attachment, proliferation and differentiation into myotubes on SM films and mats in vitro exemplifies the significance of scaffold conductivity and topography in modulating aligned myotube formation and potential of SM electrospun scaffolds in skeletal muscle tissue engineering applications.

Electroactive and antioxidant scaffolds are made with the incorporation of free radical scavenging and conducting biopolymer pigment, melanin. The composite electrospun scaffolds promotes myoblast assembly to a great extent and results in the formation of aligned high aspect ratio myotube formation. The results highlight the significance of scaffold topography along with conductivity in promoting myogenesis and the potential application of silk fibroin and melanin composite wherein the silk fibroin is optionally modified with peptides as electroactive platform for proliferation and differentiation of myoblasts into functional myotubes.

The electroactive and antioxidant properties required for skeletal muscle tissue engineering is imparted to the scaffolds in a synergistic manner using biocompatible, biodegradable, biopolymer pigment melanin and silk fibroin, wherein said silk fibroin can be optionally modified with peptides.

Thus, in an embodiment of the present disclosure, the composite comprising silk fibroin protein and melanin wherein the silk fibroin is optionally modified with peptides is a synergistic product, depicting enhanced efficacy and performance as compared to either silk fibroin or melanin alone, and depicting enhanced efficacy than is expected by mere combination of silk fibroin and melanin.

The use of biocompatible composite of the present disclosure for scaffold preparation overcomes the toxic effects associated with synthetic polymers and is more economical.

The present disclosure further relates to use of biomaterial composite and corresponding biomaterial scaffolds in regenerative medicine.

In a specific embodiment, the present disclosure provides the use of biomaterial composite comprising silk fibroin and melanin, and the corresponding biomaterial scaffold in regenerative medicine.

In another specific embodiment, the present disclosure provides the use of biomaterial composite comprising silk fibroin modified with laminin derived peptide YIGSR and/or its modified analogue GYIGSR in regenerative medicine. Said composite further comprises melanin.

In an exemplary embodiment, the present disclosure provides silk fibroin-melanin composite for differentiation of stem cells including but not limited to neural stem cells. In an embodiment, the silk fibroin is optionally modified according to the methods as described above. In a non-limiting embodiment, the composite comprising silk and melanin provides long term support and attachment of stem cells not limited to neural stem cells.

The biocompatible composite comprising silk fibroin modified with a peptide and its modified analogue has enhanced cell adhesion properties and superior stem cell differentiation ability.

In an embodiment, the present disclosure provides for use of a biomaterial composite comprising silk fibroin and melanin for the manufacture of a medicament for therapeutic application in tissue engineering, electrotherapy and regenerative medicine.

In another embodiment, the present disclosure provides for use of a biomaterial composite comprising modified silk fibroin and melanin for the manufacture of a medicament for therapeutic application in tissue engineering, electrotherapy and regenerative medicine. In an exemplary embodiment, the silk fibroin is modified with the peptide YIGSR, its modified analogue GYISR, or a combination thereof.

The present disclosure further provides for a biomaterial composite comprising silk fibroin and melanin for use in tissue engineering, electrotherapy and regenerative medicine.

The present disclosure also provides for a biomaterial composite comprising modified silk fibroin and melanin for use in tissue engineering, electrotherapy and regenerative medicine. In an exemplary embodiment, the silk fibroin is modified with the peptide YIGSR, its modified analogue GYISR, or a combination thereof.

Some of the non-limiting advantages offered by the composite and method of the present disclosure are as follows:
Enhanced antioxidant property,
Superior electroactive properties,
Improved myogenic cell differentiation,
Better cell growth and regeneration for tissue engineering techniques,
Enhanced cell adhesion property,
Proliferation of Human Mesenchymal Stem Cells (hMSCs),
Superior hMSCs differentiation into neuronal lineage,
Significance of additional flexible linker and permanent covalent modification for stem cell differentiation.
Crack free film with uniform physical properties throughout the film.
Easy accessibility of silk protein for industrial scale production of scaffolds.

The present disclosure herein below provides for certain examples for better understanding of the instant disclosure. For the purpose of the illustration, a biomaterial composite is developed; wherein the biomaterial composite is made using silk fibroin and melanin and developed into a scaffold. The silk fibroin is optionally modified/surface functionalized with a peptide and its modified analogue. However, a person skilled in the art would be aware that the illustrations provided herein are only prospective in nature and can be extrapolated to a biomaterial composite with modifications which fall under the purview of the instant disclosure.

Additional embodiments and features of the present disclosure will be apparent to one of ordinary skill in art based upon the description provided herein. The embodiments herein provide various features and advantageous details thereof in the description. Descriptions of well-known/conventional methods and techniques are omitted so as to not unnecessarily obscure the embodiments herein. Further, the disclosure herein provides for examples illustrating the above described embodiments, and in order to illustrate the embodiments of the present disclosure, certain aspects have been employed.

However, the examples used herein are intended merely to facilitate an understanding of ways in which the embodiments herein may be practiced and to further enable those of skill in the art to practice the embodiments herein. Accordingly, the following examples should not be construed as limiting the scope of the embodiments herein.

It is an object of the present disclosure to overcome or ameliorate at least one of the disadvantages of the prior art, or to provide a useful alternative.

### Examples

The following biological materials were employed in the below examples—
C2C12 mouse myoblast cell line—obtained from National Centre for Biological Science (NCBS).
Human mesenchymal stem cells (hMSCs) are procured from Institute for Regenerative medicine, Texas A&M HSC COM, USA.

### Example 1: Extraction of Silk Fibroin

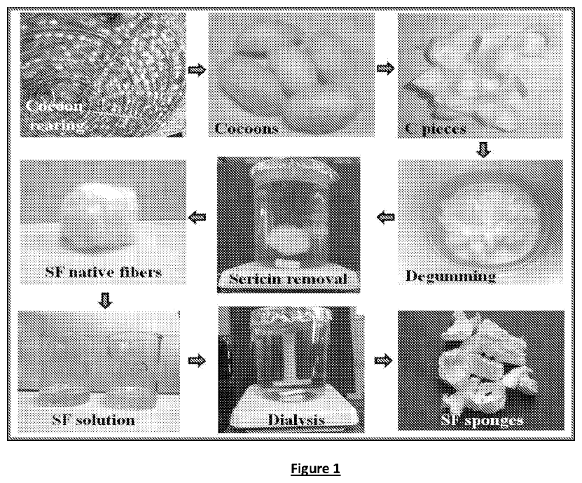

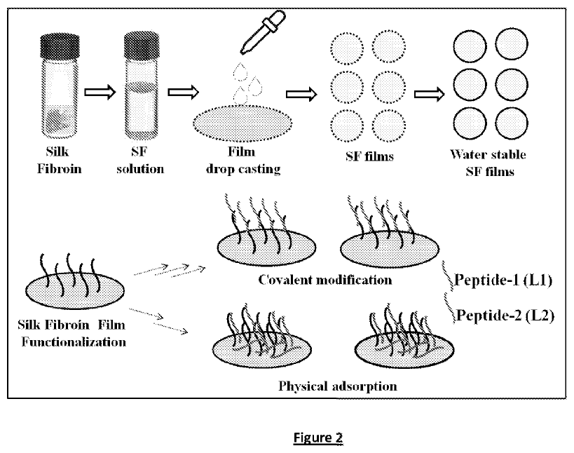

Regenerated silk fibroin solution is prepared from the mulberry silkworm Bombyx mori cocoons of CB gold variety (Mysuru, India). Briefly, the cocoons are cut into approximately 4 cm×4 cm size pieces and the water-soluble sericin is removed by boiling the cocoon pieces for 30 min in 0.02 M Na₂CO₃ solution. The resulting native silk fibroin fibers are washed thoroughly with plenty of milli-Q water to remove the water soluble sericin outer layer and dried. The obtained native silk fibroin fibers are dissolved in 9.3M LiBr (Spectrochem India) at 60° C. for 4 hours. The obtained amber-colored solution is dialyzed using an activated 14 KDa MWCO cellulose membrane (Sigma) against milli-Q water with a total of six water changes at regular intervals. The aqueous regenerated silk fibroin solution is centrifuged for 20 min at 6000 rpm, at 4° C. to remove the insoluble debris and stored at 4° C. until further use. Lyophilization of aqueous fibroin solution is carried out by initially freezing the solution using liquid N₂, followed by freeze drying at −70° C. for 14 hours to yield water-insoluble fibroin sponges. The process is shown in FIG. 1.

### Example 2: Preparation of Silk Fibroin-Melanin Composite Films

Pristine silk fibroin and melanin blend (SM; wt/wt 90:10) regenerated silk fibroin solutions made from Hexafluoro-2-propanol (HFIP) are used to fabricate biomaterial scaffold films and electrospun non-woven mats. HFIP is chosen as the solvent owing to its unique properties of dissolving most of the high molecular weight proteins at room temperature. Freestanding, flexible and peelable 2D films are fabricated by drop-casting the respective solutions onto polystyrene (PS) substrates, silk fibroin solution (6 wt %) is reconstituted from HFIP at room temperature from silk sponges. Pristine silk fibroin films (SFFs) are prepared by drop-casting the fibroin solution on to the polystyrene substrates. Drop-casted films are air-dried overnight in a fume hood and treated with 90% methanol-water mixtures for 10 min to promote β-sheet formation.

Similarly, silk-melanin composite films are prepared from the silk: melanin (wt/wt 90:10) HFIP solution (6 wt %) and subjected to methanol treatment. The treated films are dried under high vacuum to completely remove the solvents from films and stored at room temperature in airtight bags until further use.

### Example 3: Synthesis and Characterization of Peptides

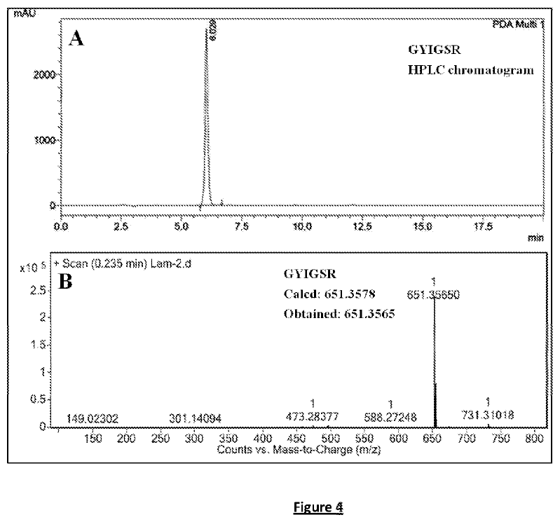

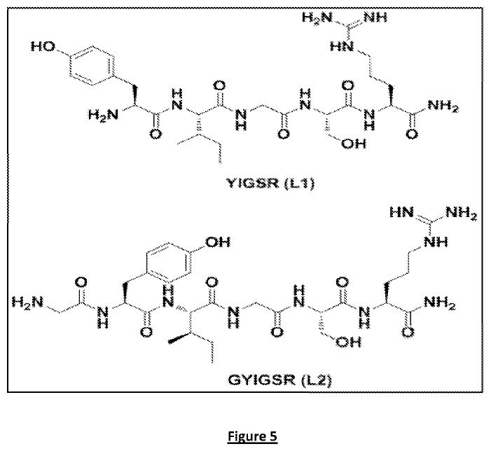

All the Fmoc amino acids with appropriate side chain protection group are obtained from Novabiochem and used as received without any further purification. Solvents and other reagents are procured either from Sigma or Spectrochem are used for the peptide synthesis. Peptides are synthesised using solid phase Fmoc-chemistry on Multisyntech Syro II automated peptide synthesiser using standard procedures. Fmoc-Rink amide resin from Novabiochem is used as solid support for the synthesis. O-(benzotriazol-1-yl)-N, N, N′, N′-tetramethyluronium hexafluorophosphate (HBTU, Novabiochem) along with N, N-diisopropylethylamine (DIPEA, Spectrochem, India) is used as activation mixture and 4 equivalents of Fmoc protected amino acids (0.5 mmol) in N, N-dimethylformamide (DMF, Spectrochem, India) is used for peptide couplings at room temperature. Peptide cleavage from the resin and side chain protection group removal is achieved by reacting the resin with cleavage cock tail (TFA:DCM:TIPS, 90:05:05) for 15 min at room temperature and the peptide solution from resin is separated using cleavage set-up of the peptide synthesiser using inert gas (dry N₂). Peptides are precipitated from the cleavage cock tail using cold diethyl ether solution for two hours. Peptides are collected by centrifugation, dried, and then purified using a reverse phase high performance liquid chromatography (LC-8A Shimadzu preparative HPLC) as shown in FIGS. 3 and 4. The peptides YIGSR and GYIGSR are obtained (FIG. 5).

The peptide purity is monitored at two wavelengths 215 nm, 275 nm for peptide content and Tyr residue respectively. High resolution mass spectrometry (HRMS, Agilent UHD Accurate-Mass Q-TOF LC/MS system) is used to further confirm the peptide mass. The results confirm the purity and identity of prepared peptides through HPLC and HRMS analysis.

### Example 4: Preparation of Modified Silk Fibroin Films (SFFs)

### Preparation of SF Films:

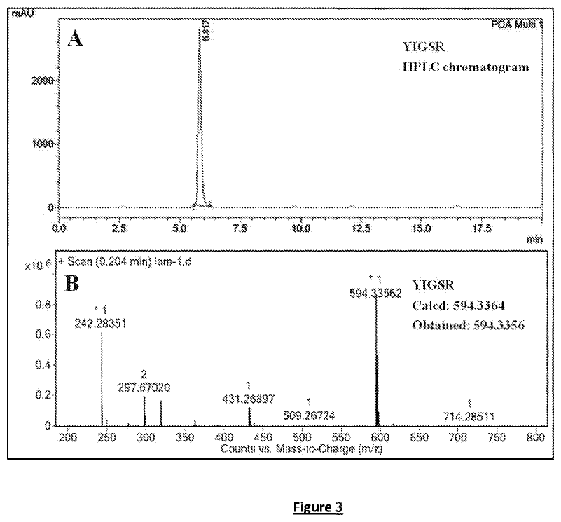

Silk fibroin sponges are dissolved in HFIP (4 wt %) at room temperature and silk fibroin films (SFFs) are prepared by drop casting method under ambient temperature and humidity. Drop casted films are air dried over night at 25° C. and treated with 90% methanol-water mixture (v/v), air dried over night at room temperature to promote the β-sheet formation and to increase the stability of films in water and in culture media. Physical and chemical modification is carried out using water stable SFFs obtained after methanol treatment as seen in FIG. 2. The peptides 1 and 2 (YIGSR and GYIGSR) (FIG. 5) are prepared using solid phase peptide synthesis (SPPS) purified and characterized using high performance liquid chromatography (HPLC) and high resolution mass spectrometry (HRMS) respectively.

The prepared silk fibroin can be modified with peptides either by physical adsorption or chemical modification (covalent attachment).

### Covalent Modification of SFFs:

The surface covalent functionalization of SFFs with prepared peptides YIGSR and GYIGSR is carried out using earlier reported method with slight modifications. SFFs are soaked in PBS (10 mM, pH 6.5) for 30 min to achieve the surface realignment of hydrophilic groups and to promote the covalent functionalization. The activation of —COOH group of Asp, Glu amino acids is achieved by treating PBS soaked SFFs with activation buffer containing 1-ethyl-3-(dimethylaminopropyl) carbodiimide hydrochloride (EDC.HCl)/N-hydroxysuccinimide (NHS) solution (0.5 mg/mL of EDC and 0.7 mg/mL of NHS in PBS buffer) for 15 min at ambient conditions to obtain the amine reactive NHS esters of Asp and Glu side chain —COOH groups. The activated NHS ester SFFs are reacted with laminin derived integrin binding peptide motifs YIGSR and GYIGSR (0.2 mg/mL) in PBS (pH 7.4) at ambient temperature for 2 h. After the reaction time SFFs are washed with PBS (pH 7.4) for 5 min, rinsed twice with PBS and once with milli-Q water to remove the buffer slats from the film surface and dried in vacuum at room temperature.

### Physical Adsorption of Peptides on SFFs:

Physical adsorption of laminin derived integrin binding peptides and whole laminin from Engelbreth-Holm-Swarm murine sarcoma basement membrane (Sigma) on SFFs surface is achieved by keeping the films in contact with YIGSR, GYIGSR (0.2 mg peptide/mL of PBS, pH 7.4) and laminin (20 μg/mL) for 4 h at 37° C. The films are then rinsed twice each with PBS and milli-Q water, dried in vacuum at room temperature.

### Example 5: Preparation of Silk Fibroin-Melanin Electrospun Mats

Extracellular matrix (ECM) mimicking non-woven, nanofibrous 3D fiber mats are prepared from pristine and melanin-blended silk fibroin using robust electrospinning technique. Electrospinning has versatility, compatibility with a wide range of synthetic and biopolymers and solvent systems, as well as easy scalability. The key electrospinning working parameters such as solvent, concentration, and composition (collectively known as solution parameters), voltage, needle diameter, flow rate, collector type and collector distance from the tip of the needle (collectively known as process parameters) and temperature and humidity (collectively known as ambient parameters) are optimized for the preparation of bead-free, uniform and reproducible fiber mats.

Silk fibroin (SF) non-woven electrospun fiber mats are made by electrospinning from its HFIP solution. SF is reconstituted from HFIP (6 wt %) and electrospinning is carried out at a voltage of 1.5 kV/cm with a flow rate of 0.8 mL/h using a 22G blunt-ended needle under ambient conditions. A custom-modified stationary surface consisting of stainless steel mesh fixed onto a stationary collector covered with aluminum foil is used to obtain large-area, easily peelable, freestanding electrospun mats. Silk/melanin composite mats are prepared similarly by electrospinning silk/melanin HFIP solution (6 wt %; 90/10). The freestanding electrospun fiber mats are peeled from the collector and treated with 90% methanol-water mixtures for 10 min to increase the aqueous stability of mats through β-sheet formation in silk. Fiber mats are dried under high vacuum and stored in an air-lock cover until further use at room temperature.

### Example 6: Characterization of Silk Fibroin-Melanin Composite

### a) Silk Fibroin and Melanin (SM) Composite:

The morphology of drop-casted 2D films and electropsun 3D fiber mat scaffolds are analyzed by field emission scanning electron microscope (FESEM, Carl Zeiss Ultra 55) at 5 kV. Briefly, the scaffolds are mounted onto SEM stubs using double-sided carbon tape and gold sputtering is carried out on the samples prior to morphological analysis. The average fiber diameter in the case of electrospun mats is determined using ImageJ software. The fiber diameter distributions are measured from 50 different locations on the SEM micrographs and the mean diameter with standard deviation is calculated. Pristine SF and composite SM films show uniform surface texture without any cracks in the structure, as shown in FIG. 9A. Crack free film signify uniform distribution of the protein in the case of pristine SF films and thorough blending of silk and melanin in the case of SM films.

SEM analysis of SF electrospun mats revealed the presence of bead-free, randomly aligned porous submicron diameter fiber networks, as shown in FIG. 9A1. The fibers are obtained with very narrow size distribution and had an average diameter of 470±45 nm, measured using ImageJ software. Similarly, SM electrospun fiber mats are obtained with uniformly distributed network of fibers with narrow size distributions. Interestingly, the formation of relatively aligned fiber mats is observed with silk-melanin blend solution under similar conditions (FIG. 9A2). The formation of bead-free and predominantly aligned fiber mats with relatively smaller fiber diameter (343±40 nm) from SM is attributed to the increased conductivity of the electrospinning solution with melanin blending. The electrospun mats are flexible for cutting or punching to attain the required scaffold dimensions for tissue engineering applications (Insets in FIG. 9A1, A2).

### b) Silk Fibroin Modified-Melanin Composite:

The surface morphology and roughness of as prepared drop-casted and methanol treated control SFFs and surface modified (both physical and chemical) SFFs is analyzed by field emission scanning electron microscope (FESEM, Carl Zeiss Ultra 55) at 5-10 kV. SF film samples are mounted onto SEM stubs using double sided carbon tape and gold sputtering is carried out on the samples prior to morphological analysis. Pristine and modified films show uniform surface texture without any cracks in the structure as shown in FIG. 6.

### Example 7: Characterization by Fourier-Transform Infrared (FTIR) Spectroscopy

The secondary structure of silk in the films and electrospun mat scaffolds are analyzed using FTIR spectroscopy with attenuated total reflection (ATR) sampling technique (GladiATR, PerkinElmer). Briefly, the spectra of as-prepared and methanol-treated scaffolds is recorded with 0.2 cm⁻¹ data interval and with a resolution of 4 cm⁻¹ using diamond crystal as substrate. All the spectra presented are the average of 64 scans in the wavelength range of 4000-400 cm⁻¹ and the data is plotted as % transmittance (% T) against wave number (cm⁻¹).

TABLE 1

| | SF Film | | SM Film | | SF mat | | SM mat | |
|---|---|---|---|---|---|---|---|---|
| Characteristic | | Methanol | | Methanol | | Methanol | | Methanol |
| absorption | As-made | treated | As-made | treated | As-spun | treated | As-spun | treated |
| Amide-I | 1650 cm−¹ | 1699 cm⁻¹ | 1642 cm⁻¹ | 1699 cm⁻¹ | 1640 cm−¹ | 1699 cm⁻¹ | 1636 cm⁻¹ | 1699 cm⁻¹ |
| | 1621 cm−¹ | 1620 cm⁻¹ | | 1619 cm⁻¹ | | 1622 cm⁻¹ | | 1623 cm⁻¹ |
| Amide-II | 1514 cm⁻¹ | 1512 cm⁻¹ | 1516 cm⁻¹ | 1512 cm⁻¹ | 1526 cm⁻¹ | 1514 cm⁻¹ | 1524 cm⁻¹ | 1515 cm⁻¹ |

FT-IR amide-I and amide-II absorption frequencies of as prepared and methanol treated SF film, SM film, SF mat and SM mats are provided in the table above.

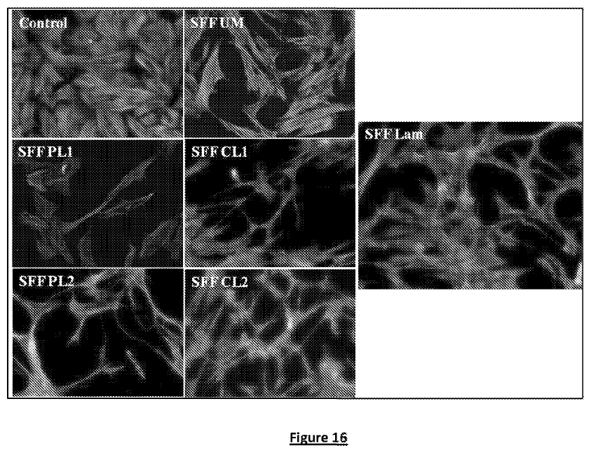

The secondary structure of the base polymer, silk fibroin in as-prepared films, electrospun mat scaffolds and the induced β-sheet formation through methanol treatment is analyzed by monitoring the amide bond vibration frequencies in IR spectrum (FIG. 8B). FTIR spectrum of as-prepared SF films show characteristic peaks at 1650 cm⁻¹, 1621 cm⁻¹ in amide-I region (C═O stretching), and 1514 cm⁻¹ in amide-II region (N—H stretching), indicating the coexistence of random helical and β-sheet conformations in the films (FIG. 13A). SF films, after methanol treatment, showed shifted peaks at 1699 cm⁻¹, 1619 cm⁻¹ and 1512 cm⁻¹ assigned to β-sheet conformation of silk (silk-II).

Similar vibrational frequency shift of characteristic amide-I and II peaks from random coil and helical conformation to β-sheet conformation is observed for SM films, and SF and SM mats with methanol treatment. The characteristic vibration frequencies of the amide bonds of as-prepared film and mats samples (1642 cm⁻¹, 1516 cm⁻¹ for SM film and 1636 cm⁻¹, 1524 cm⁻¹ for SM mat) exhibited a shift corresponding to silk-II conformation upon the methanol treatment (1699 cm⁻¹, 1619 cm⁻¹, 1512 cm⁻¹ for SM film and 1699 cm⁻¹, 1623 cm⁻¹, 1515 cm⁻¹ for SM mat). Electrospun mats exhibited more random and helical (silk I) structure in as-prepared SF and SM mats compared to the corresponding films, as seen from the amide bond absorption positions. The observed difference in the amide absorption peaks of as-prepared films and mats are attributed to the fact that the secondary structure of silk fibroin is strongly influenced by both fabrication method and the biomaterial format. FTIR analysis clearly suggested the zero interference of melanin incorporation in the secondary structure of silk fibroin in scaffolds.

FTIR analysis clearly suggests that the procedures used for the modification films have no detrimental effect on the structure of silk fibroin in film scaffolds.

### Example 8: Characterization by Contact Angle Measurement

The hydrophilicity of the pristine silk and silk-melanin composite materials is assessed by contact angle measurement (Holmarc). The influence of melanin blending on the surface wettability is evaluated by measuring the contact angles using milli-Q water and DMEM cell culture media, under ambient conditions, using the sessile drop method (Table 2). Briefly, pristine silk and silk-melanin composites using HFIP are spin-coated onto glass slides, methanol treatment is carried out and subsequently, static contact angles are measured using the sessile drop method for both water and DMEM cell culture media in triplicate. At least three sample sets (n=3) are used for all the experiments and results are expressed as mean±standard deviation for all the samples.

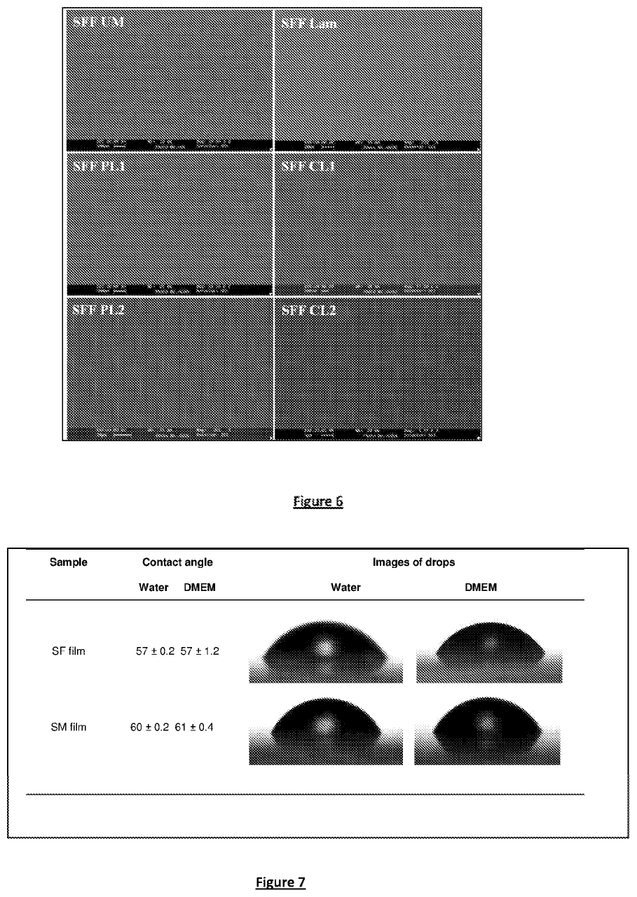

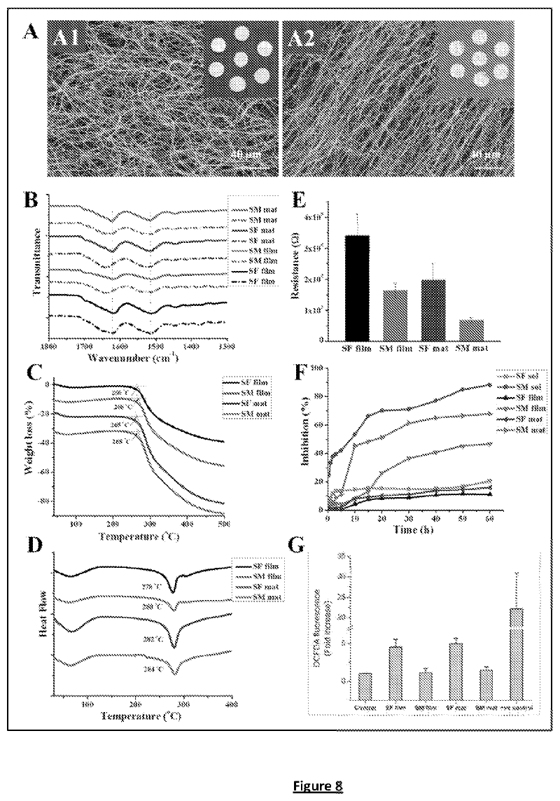

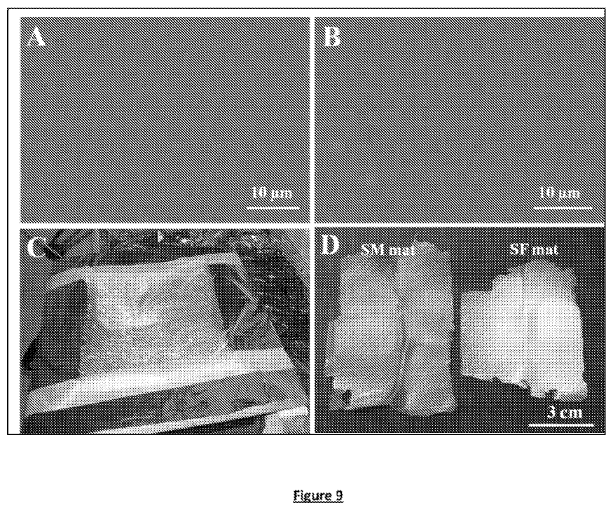

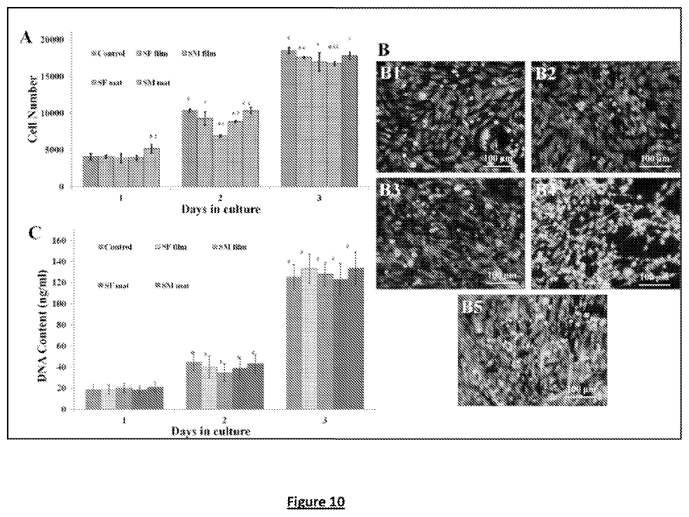

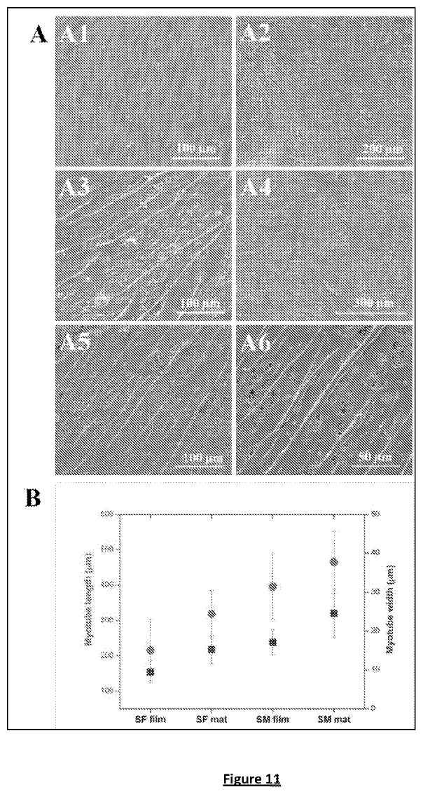

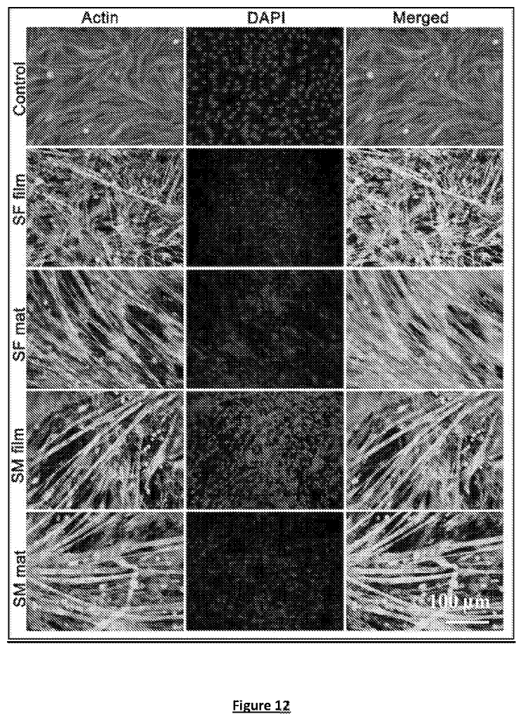

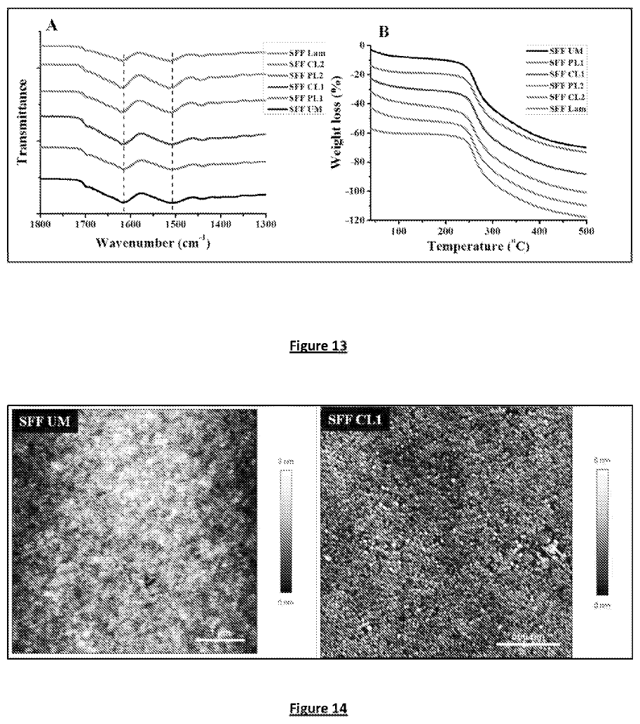

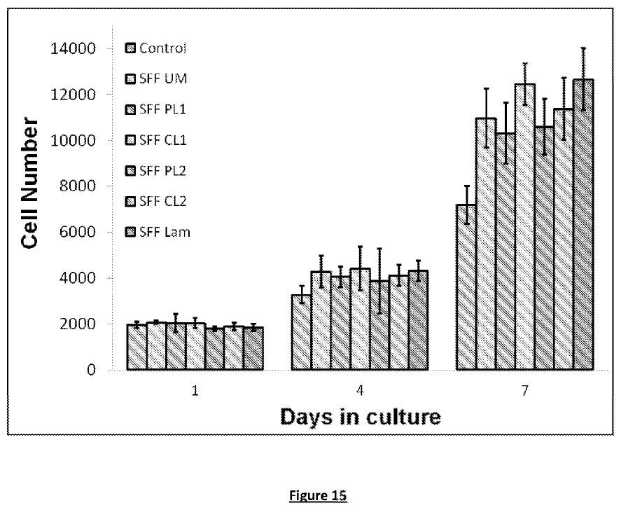

The hydrophilic properties, as reflected in their contact angles, affect cellular adhesion and proliferation on the scaffolds are provided in FIG. 7. As seen in FIG. 7, melanin blending has no profound effect on the hydrophilicity of the silk fibroin spin coated films (57±0.2 for water, 57±1.2 for DMEM) and only a marginal increase in the contact angles is observed with melanin addition, owing to the oligomeric aromatic nature of melanin (60±0.2 for water, 61±0.4 for DMEM). The scaffolds (both SF and SM) are hydrophilic, as evidenced from the wettability behavior and are well in the suitable range for cell adhesion.

TABLE 2

Contact angle

| Sample | Water | DMEM |
|---|---|---|
| SF film | 57 ± 0.2 | 57 ± 1.2 |
| SM film | 60 ± 0.2 | 61 ± 0.4 |

Hydrophilicity of pristine silk fibroin and silk/melanin composites is depicted in the table above. Contact angle values of water and DMEM droplets on the surface of SF and SM substrates are provided. The contact angle analysis values in the above table indicate the hydrophilic nature of SM substrates highlighting their usefulness for cell culture and tissue engineering applications.

### Example 9: Characterization by Atomic Force Microscopy (AFM)

The surface morphology and roughness of the drop-casted films with silk fibroin modified with peptides is measured using atomic force microscopy (JPK, NanoWizard® 3-AFM). Samples are adhered onto microscope glass slide using 2 component epoxy glue and the analysis is carried out in intermittent contact (AC) mode under ambient conditions. Pristine and covalently modified silk films show a very smooth surface and only a marginal increase in the surface roughness with covalent modification (RMS=637 μm for SFF UM to RMS=1.25 nm for SFF CL1) as shown in FIG. 14.

### Example 10: Characterization by Analyzing Thermal Stability

The thermal decomposition behavior of the SF and SM films and electrospun fiber mats/scaffolds (SF mat and SM mat) is evaluated using thermo gravimetric (TG; Mettler, TGA/DSC 2) and differential scanning calorimetric (DSC; TA DSC, Q2000) analysis. The initial weight loss around 100° C. for all the scaffolds is due to the loss of water. TG curves of SF film, SM film, SF mat and SM mat shows decomposition temperature at 266° C., 268° C., 265° C. and 268° C., respectively. The decomposition temperature of melanin-incorporated films and electrospun mats is higher than the corresponding pristine SF scaffolds. The higher thermal stability, as reflected in decomposition temperatures of SM scaffolds, is attributed to the increased stability of scaffolds with the incorporation of melanin with the aromatic backbone.

TGA measurement is carried out by heating the scaffolds at 5° C./min in the temperature range of 40° C. to 600° C. under the continuous dry nitrogen flow of 20 mL/min. The higher thermal stability of melanin-incorporated scaffolds is further confirmed by carrying out DSC analysis. DSC curves show higher degradation temperature for SM film and SM mat at 280° C. and 284° C., respectively, than that of the SF film and SF mat at 278° C. and 282° C., respectively (FIG. 8D). DSC is carried out at a heating rate of 5° C./min in the temperature range of 40° C. to 400° C. under the continuous 20 mL/min nitrogen gas flow. TG graphs of SF and SM scaffolds are shown in FIG. 8C. TG graphs of all silk films are shown in FIG. 13B. TG curves of all silk films show decomposition temperatures >260° C. indicating the high thermal stability of silk films.

### Example 11: Conductivity Measurements

The electrical conducting property of silk and melanin composites in comparison with silk alone is evaluated using a two-point resistivity probe at 25° C. and under humid conditions. Electrical contacts of about 100 nm thickness on the glass slides are made by thermal evaporation of gold using a shadow mask. Samples (films and electrospun mats) for conductivity measurement are prepared on glass slides containing patterned gold electrodes by spin-coating and electrospinning from the corresponding silk fibroin and silk-melanin solutions. The electrical properties of scaffolds is measured for methanol-treated samples under humid conditions to mimic the cell culture environment using Keithley 2420 and a two-point resistivity probe according to previously reported method. SM spin-coated and electrospun samples exhibit a characteristic linear variation of current with voltage, as indicated from I-V curves compared to the pristine SF scaffolds. The slope of 1-V curve becomes more prominent in the case of SM samples under the studied humid conditions.

Sheet resistance values for SF, SM spin-coated and electrospun scaffolds measured under physiological conditions are shown in FIG. 8E. Sheet resistance values are used to estimate the improvement in electroactive nature of scaffolds with melanin incorporation, instead of conductivity values owing to the porous nature of electrospun scaffolds. The spin-coated SM samples show half of the sheet resistance values compared to the corresponding SF spin-coated samples, highlighting the contribution from melanin. Electrospun SM scaffolds display further decrease in the sheet resistance values under the same conditions and as much as 2.5 times less sheet resistance is observed (1.64E+05Ω for SM films to 6.72E+04Ω for SM mats, FIG. 8E). The observed lowest sheet resistance values for SM mats as compared to SM films and SF scaffolds is attributed to the porous nature, increased hydration and hydration-dependent conductivity of melanin.

### Example 12: Antioxidant Activity

The radical scavenging capacity of silk-melanin solution and scaffolds is evaluated against the inhibition of lipophilic radicals (1,1-diphenyl-2-picrylhydrazyl (DPPH)) over a time period of 60 hours. The results of antioxidant activity are shown in FIG. 8F as percentage inhibition of radicals by scaffolds with time. The intrinsic antioxidant activity of silk-melanin solutions and scaffolds is evaluated against the inhibition of lipophilic radical (1,1-diphenyl-2-picrylhydrazyl (DPPH)) ions using previously reported protocol with slight modifications. The efficiency of silk-melanin solutions and scaffolds to inhibit the DPPH (100 μM) radicals in aqueous ethanol is evaluated by incubating 60 μl of solutions and scaffolds containing equal amount of pure and composite silk material and measuring the absorbance at 490 nm using a microplate reader (Eppendorf BioSpectrometer® AF2200).

The antioxidant efficacy of solutions and scaffolds is expressed as percentage of free radical inhibition as a function of incubation time. Highest radical scavenging activity is shown by silk-melanin solutions with 88% inhibition, followed by SM mats and SM films with 68% and 46% of radical inhibition, respectively.

Notably, SM solutions show rapid radical scavenging activity compared to the corresponding scaffolds, while the film and mat scaffolds of SM exhibit persistent activity over a prolonged time period compared to SM solutions, a desired property for cell culture applications. The rapid radical scavenging activity of SM solutions is attributed to the increased availability of SM material for radical inhibition. Silk fibroin solutions, films and electrospun mats show very little antioxidant activity as compared to SM scaffolds under the studied experimental conditions. The consistent radical scavenging activity of SM solutions and scaffolds is assigned to the strong antioxidant property of incorporated melanin.

### Example 13: Cell Culture

### a) Silk Fibroin and Melanin Composite

C2C12 mouse myoblast cell line obtained from National Centre for Biological Science (NCBS) is used in the in vitro cell culture experiments. Cryo-preserved cells are revived and expanded in Dulbecco's modified Eagle's medium (DMEM; Invitrogen) supplemented with 20% Fetal Bovine Serum (FBS; Invitrogen), 1% antibiotic antimycotic solution (Sigma) and 2 mM L-glutamine (Invitrogen). Cell cultures are maintained at 37° C. using a humidified CO₂ incubator (Sanyo, MCO-18AC, USA) and the culture media is changed every alternate day of culture. Cells are detached from the tissue culture flasks after reaching 70-80% of the confluency using 0.05% trypsin-EDTA (Invitrogen) and sub-cultured for further use. All the cell culture experiments are carried out using cells from passages 3-7.

b) Silk Fibroin Modified with Peptide

Human mesenchymal stem cells (hMSCs) are procured from Institute for Regenerative medicine, Texas A&M HSC COM, USA and all the experiments involving hMSCs are carried out with the prior approval from the Institutional Committee for Stem Cell Research and Therapy (IC-SCRT), IISc, Bangalore. The cells from cryopreserved stock are revived and grown in complete growth medium containing alpha modified Eagle's medium (αMEM; Invitrogen) supplemented with 20% fetal bovine serum (MSC FBS; Invitrogen), 1% antibiotic antimycotic solution (Sigma) and 2 mM L-glutamine (Invitrogen). Cell cultures are maintained at 37° C., 95% humidity and 5% CO2 using a humidified CO2 incubator (Sanyo, MCO-18AC, USA) and the culture media is changed every alternate day of culture. Cells are detached from the tissue culture flasks upon reaching 70-80% of the confluency using 0.05% trypsin-EDTA (Invitrogen) and harvested by neutralizing with the complete media and centrifugation at 1500 rpm for 5 min. The cells are then sub-cultured for further use as required.

### Example 14: Cell Viability Studies

The biocompatibility of scaffolds comprising silk fibroin-melanin for muscle tissue engineering is evaluated by measuring the viability, cytotoxicity and proliferation of myoblasts seeded onto the scaffolds through MTT, LIVE/DEAD and cell proliferation assays, respectively.

i) MTT Assay

### a) Silk Fibroin and Melanin Composite

The viability of cells grown on the scaffolds is evaluated for their mitochondrial reductase activity of live cells using MTT assay. The metabolic activity of mouse myoblast cells (C2C12) on different SF and SM scaffolds is evaluated after 24, 48 and 72 hours using MTT assay. Cell viability test is performed using MTT (3(4,5-dimethylthiazol-2-yl)-2,5-diphenyltetrazolium bromide, Sigma) assay on pristine and silk-melanin composite scaffolds (films and electro spun mats). MTT assay is one of the best quantitative assays to determine the metabolic activity of cells. MTT interacts directly with the mitochondria of live cells and the resulting change in the optical density gives the number of live cells. Approximately 5×10³ cells/mL of C2C12 myoblast cells are seeded onto the sterilized samples placed in 24 well plates and incubated for 1, 2 and 3 days in humidified CO₂ incubator at 37° C.

After the desired incubation period, the medium in the well plate is removed and washed twice with PBS, followed by addition of 15% MTT reagent (Sigma) prepared in DMEM (without phenol red) for 4 hours. The purple-colored formazan crystals formed are solubilized using high purity DMSO (Merck) and the intensity of color is measured by recording the optical density at 595 nm using a microplate reader (iMark, Bio-rad laboratories, India). The measured intensity of formed formazan crystals is the direct measure of the number of viable and metabolically active C2C12 mouse myoblast cells on the scaffolds. As shown in FIG. 10A, a consistent yet statistically significant increase in the number of viable cells is observed from day 1 to day 3. There is no significant difference in the cell number on the scaffolds on all the days.

### b) Peptide Modified Silk Fibroin

The suitableness of scaffolds for tissue engineering and regenerative medicine applications is evaluated by measuring the viability of hMSCs seeded on to the films through MTT assay. The cytocompatibility of functionalized silk films is evaluated by measuring the metabolic activity/viability and proliferation of hMSCs seeded onto 16 mm diameter circular SFFs with an average thickness of 15 μm. Cell viability test is performed using MTT (3(4,5-dimethylthiazol-2-yl)-2,5-diphenyltetrazolium bromide, Sigma) assay on pristine and functionalized silk films. MTT interacts with mitochondria of live cells and the resulting change in the optical density gives the number of live cells, hence the percentage of viable cells as compared to TCPs control. Approximately 104 cells/mL of hMSCs are seeded on the sterilized films placed in 24 well plates and incubated for 1 (24 hr), 4(72 hr) and 7(168 hr) days in humidified CO2 incubator. The cell culture medium in the wells are removed and washed twice with PBS after stipulated period of culture, 15% MTT reagent (Sigma) prepared in DMEM (without phenol red) is added and incubated for 4 h at 37° C. The purple colored formazan crystals formed is solubilized using high purity DMSO (Merck) and the intensity of colour is measured by recording the optical density at 595 nm using a microplate reader (iMark, Bio-rad laboratories, India). The measured intensity of formed formazan crystals is the direct measure of number of viable and metabolically active hMSCs on the silk film scaffolds.

All the SFFs scaffolds show good cytocomaptibility and no significant cell death is observed on any silk scaffold with respect to TCPs control after day 1. A consistent and statistically significant increase in the number of viable hMSCs as compared to the TCPs control is observed for both 72 and 168 h cultures as shown in FIG. 8. The markedly increased cell number on SFFs on day 7 indicates that the scaffolds promote hMSCs adhesion and proliferation to a great extent.

The suitableness of scaffolds for tissue engineering and regenerative medicine applications is evaluated by measuring the viability of hMSCs seeded on to the films through MTT assay. The cytocompatibility of functionalized silk films is evaluated by measuring the metabolic activity/viability and proliferation of hMSCs seeded onto 16 mm diameter circular SFFs with an average thickness of 15 μm. Cell viability test is performed using MTT (3(4,5-dimethylthiazol-2-yl)-2,5-diphenyltetrazolium bromide, Sigma) assay on pristine and functionalized silk films. MTT interacts with mitochondria of live cells and the resulting change in the optical density gives the number of live cells, hence the percentage of viable cells as compared to TCPs control. Approximately 104 cells/mL of hMSCs are seeded on the sterilized films placed in 24 well plates and incubated for 1 (24 hr), 4(72 hr) and 7(168 hr) days in humidified CO2 incubator. The cell culture medium in the wells is removed and washed twice with PBS after stipulated period of culture, 15% MTT reagent (Sigma) prepared in DMEM (without phenol red) is added and incubated for 4 h at 37° C. The purple colored formazan crystals formed is solubilized using high purity DMSO (Merck) and the intensity of colour is measured by recording the optical density at 595 nm using a microplate reader (iMark, Bio-rad laboratories, India). The measured intensity of formed formazan crystals is the direct measure of number of viable and metabolically active hMSCs on the silk film scaffolds. All the SFFs scaffolds show good cytocomaptibility and no significant cell death is observed on any silk scaffold with respect to TCPs control after day 1. A consistent and statistically significant increase in the number of viable hMSCs as compared to the TCPs control is observed for both 72 and 168 h cultures as shown in FIG. 15. The markedly increased cell number on SFFs on day 7 indicates that the scaffolds promote hMSCs adhesion and proliferation to a great extent.

II) Live/Dead Assay

The cytotoxicity of pristine and silk-melanin composite scaffolds (films and mats) on myoblasts is calculated by LIVE/DEAD assay. Fluorescein diacetate (FDA) and propidium iodide (PI) dye combination is used for staining the live and dead cells, respectively. Esterases present in the live cells catalyze the de-esterification of non-fluorescent FDA to green fluorescent fluorescein and impart green fluorescent color to the live cells. The red nuclei in the fluorescent images represent the dead cells arising from PI intercalation with nuclear DNA of dead or cell membrane-compromised cells. Myoblasts on scaffolds, after the stipulated period of culture, are washed with 1×PBS and stained with 1 mL of FDA (25 mg/mL) for 15 min at 37° C. and with 1 mL of PI (10 mg/mL) for 5 min at room temperature. Samples are washed twice with 1×PBS and myoblast LIVE/DEAD ratio is assessed by fluorescence microscope imaging within 15-20 min post the staining procedure. Representative images of cells cultured on SF and SM scaffolds stained with fluorescein diacetate (FDA)-propidium iodide (PI) dye combination is shown in FIG. 10B. A high fraction (>99%) of FDA-stained cells, with only few nuclei stained with PI, is observed on the scaffolds, indicating excellent biocompatibility of the scaffolds.

Homogeneous distribution of cells on the surface of films and infiltration through the pores in the case of electrospun mats is observed for both SF and SM scaffolds. The evidence for infiltration of cells into the electrospun mats is seen in FIG. 10 B3, B5, when the focus is on surface of the mat, the interior cells are out of focus. In addition to infiltration through pores, the cells grown on electrospun mats show distinct stretched morphological behavior, unlike the cells cultured on films. Melanin incorporation further assists in extended morphological stretching and alignment of cells on SM mats as compared to the electrospun mats from SF alone (FIG. 10 B3, B5).

III) Cell Proliferation Studies

The proliferation of myoblasts is further evaluated by quantifying the total DNA content of cells cultured on pristine and composite silk scaffolds after 24, 48 and 72 hours using picogreen assay.

Picogreen assay is used to measure the proliferation of myoblasts (FIG. 5) by measuring the total DNA content of the cells cultured on pristine and composite silk scaffolds. Quanti-iT Picogreen dsDNA assay kit (Invitrogen) is used to quantify the total DNA content, as per the manufacturer's protocol. After 1, 2 and 3 days of myoblast culture, the samples are washed in IX PBS and lyzed with 250 mL of 0.1% Triton-X for 10 min. Equal volumes of 1×TE buffer and 100 mL of Picogreen working reagent (1: 300 dilution of the stock) are added in a 96-well plate to the cell lysate. The fluorescence intensities are recorded after 5 min incubation using a multi-mode plate reader (Eppendorf AF2200) with excitation and emission wavelengths of 485 and 535 nm, respectively. Standard curve of known dsDNA (ng/mL) is used to calculate the DNA content from the samples.

As shown in FIG. 10C, a marked increase in the DNA content of myoblasts grown on each scaffold after 24, 48 and 72 hours is observed. There is no significant difference in the DNA content of cells grown on all the scaffolds, indicating the excellent cyto-compatibility of scaffolds. The proliferation studies have demonstrated that silk scaffolds are equally compatible with myoblast cells as with the control substrates (TCPS). The topography of scaffolds (films and mats) has the least effect on cell viability and proliferation rate, but rather has a profound effect on the morphological alteration of the myoblasts with an additive effect imparted by melanin incorporation.

### Example 15: Oxidative Stress Study in Cells

Oxidative stress developed in myoblasts during the culture impairs myogenic differentiation and even inhibits myogenesis at higher concentrations. Further, the increased oxidative stress plays a detrimental role during muscular pathologies by altering the muscle functionality and even leads to cell death. The protective role of SM scaffolds against the oxidative stress and consequent lethal effect on myoblast culture and differentiation is therefore evaluated by measuring the intracellular reactive oxygen species (ROS) levels of myoblasts cultured on silk scaffolds using 5-(and-6)-chloromethyl-2′,7′-dichlorodihydrofluorescein diacetate (CM-H2DCFDA) assay.

The reduction of cellular oxidative stress in the myoblasts cultured on SF and SM scaffolds is estimated using 5-(and-6)-chloromethyl-2′,7′-dichlorodihydrofluorescein diacetate (CM-H2DCFDA) ROS indicator dye (Invitrogen). Myoblasts on scaffolds, after 48 hours of culture, are incubated with CM-H2DCFDA (10 μM) for 30 min at 37° C. in dark. Samples are washed thrice with IX PBS and subsequently, the intrinsic ROS level of cells on different scaffolds is estimated by measuring the fluorescence intensity. Fluorescence changes are monitored at 535 nm with the excitation at 485 nm.

FIG. 8G shows the DCFDA fluorescence from myoblasts cultured on SF and SM scaffolds for 48 hours. A twofold decrease in the DCFDA fluorescence intensity is observed from the myoblasts cultured on SM scaffolds (films and mats) as against the myoblasts cultured on the pristine SF scaffolds. Myoblasts cultured on TCPS acted as control to observe the oxidative stress of myoblasts on scaffolds, and myoblasts on TCPS with extrinsically added hydrogen peroxide (H₂O₂) serves as the positive control for DCFDA fluorescence. The observed decrease in DCFDA fluorescence on SM scaffolds (films and mats) further asserts the protective antioxidant activity of melanin/silk fibroin scaffolds against the basal ROS. Thus, melanin blending with SF imparts a unique protective function against lethal oxidative stress to the SM scaffolds.

### Example 16: Myoblast Differentiation Studies

Myogenesis is induced by subjecting the myoblasts cultured on SF and SM scaffolds to serum starvation conditions after reaching confluency. Myoblasts start fusing under serum starvation condition and result in the formation of multi-nucleated myotubes. The high aspect ratio of long myotubes formed on SM scaffolds after 7 days of culture (3 days in differentiation media) are visualized using SEM and the corresponding images are shown in FIG. 11A.

Myoblasts cultured on TCPS control do not show any myogenic differentiation and remain as individual cells even after 3 days of culture in differentiation medium. SF scaffolds with low or no conductivity result in the formation of shorter and less prominent myotubes without any regular alignment. The formation of relatively better myotubes on SF electrospun mats, as compared to the SF films, is attributed to the topographical cues of SF mats mimicking extracellular matrix (ECM), highlighting the significance of topography in myoblast differentiation. SM films support the myogenesis to some extent and formation of short and random myotubes as compared to the SF films and mats, highlighting the instructive role of conducting melanin in myogenesis.

Differentiation of myoblasts on scaffolds into multi-nucleated myotubes is induced by replacing complete media with DMEM containing 1% FBS after the culture attained 90% confluence. Cells are then grown under serum starvation conditions for the complete fusion of myoblasts to give rise to myotubes. The formation of myotubes on the pristine and composite silk scaffolds is analyzed by observing the morphological features under scanning electron microscope and using fluorescence microscope after actin staining. For SEM imaging, the samples are washed with PBS and fixed with 1.5% glutaraldehyde (Loba Chemie, India) in PBS for 30 min at 4° C. A series of ethanol washes (30, 50, 70, 90, and 100%) is done subsequently to dehydrate the samples completely. The dried samples are then sputter-coated (Vacuum Tech, Bangalore, India) with gold and observed under scanning electron microscope (FEI Inspect). Actin filaments are visualized under fluorescence microscope (Nikon Eclipse, model LV100D, Japan) after staining with Alexa Fluor 488-Phalloidin (Invitrogen) for 20 min and the nuclei are counterstained with Hoechst stain 33342 (Invitrogen) (FIG. 12).

The best myogenic differentiation is observed on SM electrospun mats, which combine both the properties of scaffold conductivity and ECM mimicking topography. After 7 days of culture, myoblast cells on SM mats differentiate into well-defined and aligned myotubes with very good myotube aspect ratio (length to width ratio). The extent of myogenesis on different scaffolds is further quantified by measuring the average myotube length and width after 3 days of culture in differentiation medium. FIG. 11B shows the calculated average length and width of myotubes on SF and SM scaffolds. A marked increase in myotube length and width on SM electrospun mats from the quantitative calculation further highlights the potential of SM electrospun mats for skeletal tissue engineering. The myotube assembly is further analyzed by staining the actin cytoskeleton with Alexa Fluor 488-Phalloidin and by counterstaining the nucleus with Hoechst stain.

FIG. 12 shows the fluorescence images of myoblast cells that have proliferated on SF and SM scaffolds along with the control, visualized after 3 days of culture (in differentiation media). As can be seen in FIG. 12, after 3 days of culture in serum starvation, a confluent monolayer of individual myoblast cells is observed on the control TCPS. Concurrently on all silk scaffolds, the cells are fused to form multi-nucleated myotubes (SM mat>SM film>SF mat>SF film>TCPS).

Especially, the myotubes formed on SM mats are numerous, with high aspect ratio, and are often branched. There is no appreciable difference in the myotube number and aspect ratio between SM films and mats, except for the presence of multiple branching in SM mats. Besides, the number of myotubes formed on SM scaffolds is higher than SF scaffolds, suggesting improved myotube organization and differentiation upon incorporation of melanin. The multinucleated myotubes are more aligned on mats than on films, forming long myofibers. Overall, the myoblast differentiation studies confirm the crucial role of topography and conductivity of biomaterial scaffolds in determining the myoblast cell fate. Myotube length and width is calculated from SEM images using ImageJ software and all the results presented are the average of 5 calculations.

### Statistical Analysis

Results are reported as mean±standard deviation and the statistical analysis is carried out using SPSS-16.0 (IBM, USA) software. At least three sample sets (n=3) are used for all the experiments and is repeated at least thrice. Values of p<0.05 are considered as statistically significant.

The present disclosure relates to biomaterial composites comprising silk fibroin and melanin, scaffolds made of the composite and medical/regenerative applications of the scaffolds. The combined synergistic action of silk fibroin and melanin provides for a highly efficient scaffold material. The present disclosure relates to successfully fabricated biocompatible, pristine and melanin composite silk fibroin biomaterial scaffolds with antioxidant and electroactive properties and further explores their potential use in skeletal muscle tissue engineering.

Melanin incorporation shows a distinctly positive effect on the scaffold properties and improved the myogenic differentiation of myoblasts into myotubes in vitro. Silk/melanin composite scaffolds showed strong antioxidant properties, helping in the reduction of the intracellular ROS levels and, hence, the oxidative stress. Silk fibroin/melanin composite electrospun fiber mats support the proliferation of mouse myoblast C2C12 cells and induce better differentiation into aligned high aspect ratio myotubes compared to the corresponding films, highlighting the significance of both topography and conductivity of scaffolds in muscle tissue engineering. This study is the first attempt to impart dual functionality of antioxidant property and electrical conductivity to biomaterial scaffolds made exclusively from biopolymers for skeletal muscle tissue engineering.

### Example 17: hMSCs Differentiation Studies

Differentiation of hMSCs on modified SFFs is studied in the presence of neuronal lineage inducing biochemical cue, retinoic acid (RA). The differentiation of hMSCs into neural like cells is induced by the addition of RA to αMEM culture media. The formation of neural like cells on the pristine and modified SFFs is analyzed by observing the morphological features of hMSCs on different SFFs using fluorescence microscope after staining the actin cytoskeleton as shown in FIG. 9. Actin filaments are visualized under fluorescence microscope (Nikon Eclipse, model LV100D, Japan) after staining with Alexa Fluor 488-Phalloidin (Invitrogen) for 20 min and by nucleus counterstaining (19 counterstained) with Hoechst stain 33342 (Invitrogen) after 7 days of culture. As can be seen in FIG. 16, a confluent monolayer of well spread cells of typical characteristic of hMSCs is observed. hMSCs cultured on unmodified or pristine SFFs (SFF UM) show slightly elongated morphology as compared to TCPs control. SFFs functionalized by YIGSR(L1) by physical adsorption (SFF PL1) show morphological features similar to SFF UM except for the presence of small population of hMSCs with morphology. SFFs covalently functionalized with YIGSR (SFF CL1) show cell population having stretched morphology with good interconnectivity among neighboring cells. Silk films functionalized by physical adsorption (SFF PL2) and covalent bond formation (SFF CL2) with GYIGSR (L2) show mostly stretched morphology and maximum interconnectivity with neighboring cells. Most of the cells on SFF CL2 are elongated and well interconnected when compared to SFF CL1 and further show similar behavior as scaffolds coated with whole laminin protein (SFF Lam). The results indicate that, the addition of a flexible linker in the form of Gly to the N-terminus of lamnin (31 sequence has a profound effect in driving the hMSCs differentiation into neuronal lineage similar to the whole laminin protein. Further, covalently attached GYIGSR(L2) show better results as compared to the physically coated samples, highlighting the significance of covalent modification in controlling the differentiation fate of stem cells.

Obviously, many modifications and variations are possible in light of the above teachings. It is therefore to be understood that the claimed subject matter may be practiced otherwise than as specifically described. Any reference to claim elements in the singular, for example, using the articles “a,” “an,” “the,” or “said” is not to be construed as limiting the disclosure.

The foregoing description of the specific embodiments of the present disclosure reveals the general nature of the embodiments herein that others can readily modify and/or adapt for various applications by applying current knowledge. Such specific embodiments of the disclosure, without departing from the generic concept, and, therefore, such adaptations and modifications should and are intended to be comprehended within the meaning and range of equivalents of the disclosed embodiments.

It is also to be understood that the phrases or terms employed herein are for the purpose of description and not intended to be of any limitation. Therefore, while the embodiments in this disclosure have been described in terms of preferred embodiments, those skilled in the art will recognize that the embodiments herein can be practiced with modifications, within the spirit and scope of the embodiments as described in the present disclosure. Throughout the present disclosure, the word “comprise”, or variations such as “comprises” or “comprising” wherever used, are to be understood to imply the inclusion of a stated element, integer or step, or group of elements, integers or steps, but not the exclusion of any other element, integer or step, or group of elements, integers or steps.

With respect to the use of substantially any plural and/or singular terms in the present disclosure, those of skill in the art can translate from the plural to the singular and/or from the singular to the plural as is considered appropriate to the context and/or application. The various singular/plural permutations may be expressly set forth herein for the sake of clarity.

Any discussion of documents, acts, materials, devices, articles and the like that has been included in this specification is solely for the purpose of providing a context for the present disclosure. It is not to be taken as an admission that any or all of these matters form a part of the prior art base or were common general knowledge in the field relevant to the present disclosure as it existed anywhere before the priority date of this application.

While considerable emphasis has been placed herein on the particular features of this disclosure, it will be appreciated that various modifications can be made, and that many changes can be made in the preferred embodiments without departing from the principles of the disclosure.

These and other modifications in the nature of the disclosure or the preferred embodiments will be apparent to those skilled in the art from the disclosure herein, whereby it is to be distinctly understood that the foregoing descriptive matter is to be interpreted merely as illustrative of the disclosure and not as a limitation.

1. A biomaterial composite comprising silk protein and melanin.

2. The biomaterial composite as claimed in claim 1, wherein the silk protein is silk fibroin, spider silk or a combination thereof, preferably silk fibroin.

3. The biomaterial composite as claimed in claim 1, wherein the silk fibroin and the melanin are in amount ranging from about 95:05 (wt/wt) to 80:20 (wt/wt), preferably 90:10 (wt/wt).

4. The biomaterial composite as claimed in claim 1, wherein the silk fibroin is optionally modified with peptide.

5. The biomaterial composite as claimed in claim 4, wherein the peptide is a laminin derived peptide selected from a group comprising YIGSR, GYIGSR and a combination thereof.

6. The biomaterial composite as claimed in claim 1, wherein the composite is in a format selected from a group comprising film, mat, membrane, gel, sponge and combinations thereof.

7. A method of preparing the biomaterial composite of claim 1 comprising steps of:
a) obtaining silk protein sponges; and
b) treating the silk protein sponges with melanin in presence of a solvent to obtain the composite.

8. The method as claimed in claim 7, wherein the silk protein is silk fibroin; and wherein the silk fibroin and melanin are in amounts ranging from about 95:05 to 80:20.

9. The method as claimed in claim 7, wherein the method is carried out at a temperature ranging from about 25° C. to 30° C., and for a time period ranging from about 48 hours to 96 hours.

10. The method as claimed in claim 7, wherein the solvent is selected from a group comprising Hexafluoro-2-propanol (HFIP), formic acid and a combination thereof.

11. The method as claimed in claim 7, wherein the composite is in a format selected from a group comprising film, mat, membrane, gel, sponge and combinations thereof; wherein the composite film is obtained by drop casting the composite solution obtained in step (b) of claim 7 followed by treatment with a solvent and drying; and wherein the composite mat is obtained by electrospinning the composite solution obtained in step (b) of claim 7.

12. The method as claimed in claim 7, wherein the silk protein is optionally modified with peptide; and wherein the peptide modified silk protein is prepared by physical adsorption of the peptide or covalent modification of the silk protein with the peptide wherein said covalent modification comprises covalent peptide bond formation with N-terminus of the peptide to aspartic acid, glutamic acid side chain acid groups of the silk protein, or a combination thereof.

13. A biomaterial scaffold comprising the composite of claim 1.

14. A peptide modified silk fibroin, wherein the peptide is a laminin derived peptide GYIGSR.

15. A method of preparing peptide modified silk fibroin of claim 14, said method comprising physically adsorbing the peptide on the silk fibroin or covalent modification of the silk fibroin with the peptide wherein said covalent modification comprises covalent peptide bond formation with N-terminus of the peptide to aspartic acid, glutamic acid side chain acid groups of the silk fibroin, or a combination thereof.

16. (canceled)

17. (canceled)

18. A method of repairing or regenerating a biological tissue, said method comprising a step of contacting:
(a) a biomaterial composite comprising silk protein and melanin, or
(b) a biomaterial scaffold comprising said composite, or
(c) a peptide modified silk fibroin wherein the peptide is a laminin derived peptide GYIGSR, with an existing tissue or cells to repair or regenerate the biological tissue.

19. The method as claimed in claim 18, wherein the tissue or cells are selected from a group comprising stem cells, brain cells, cranial tissue, nerve tissue, spinal disc, lung, cardiac muscle, skeletal muscle, bone, cartilage, tendon, ligament, liver, kidney, spleen, pancreas, bladder, pelvic floor, uterus, blood vessel, breast, skin and combinations thereof.

20. A method of treating a medical condition in a subject, said method comprising a step of implanting:
(a) the biomaterial scaffold of claim 13, or
(b) a biomaterial scaffold comprising a peptide modified silk fibroin wherein the peptide is a laminin derived peptide GYIGSR, into the subject to treat the medical condition.

21. The method as claimed in claim 20, wherein the medical condition is selected from a group comprising rheumatic disease, neurodegenerative disease, cardiovascular disease, wound healing and combinations thereof.
